(19) United States
(12) Patent Application Publication
(10) Pub. No.: US 2026/0274950 A1
(43) Pub. Date: Sep. 17, 2026

(54) CD3-Targeting Antibody, Bispecific Antibody And Use Thereof

(71) Applicant: HARBOUR BIOMED (SHANGHAI) CO., LTD, Shanghai (CN)

(72) Inventors: Yun HE, Shanghai (CN); Lei SHI, Shanghai (CN)

(21) Appl. No.: 19/695,504

(22) Filed: Jun. 2, 2026

Related U.S. Application Data
(63) Continuation of application No. 17/764,284, filed on Sep. 29, 2020, now Pat. No. 12,668,631.

(30) Foreign Application Priority Data
Sep. 30, 2019 (CN) 201910941328

(51) Int. Cl. C07K 16/28; A61P 35/00

(52) U.S. Cl. CPC C07K 16/2809; A61P 35/00; C07K 16/2803; C07K 16/2827; C07K 2317/24; C07K 2317/31; C07K 2317/56; C07K 2317/622

(57) ABSTRACT

Disclosed are a CD3-targeting antibody, a bispecific antibody and the use thereof. The CD3-targeting antibody reduces the toxicity caused by cytokine release syndrome, and the bispecific antibody prepared therefrom is stable and has the ability to bind to T cells, and also reduces the difficulty of producing.

### CROSS REFERENCE TO RELATED APPLICATIONS

This application is a continuation application of application Ser. No. 17/764,284, filed on Mar. 28, 2022, which is a National Phase of Application No. PCT/CN2020/118606, filed on Sep. 29, 2020, which claims priority to Chinese patent application 2019109413286 filed on Sep. 30, 2019, the contents of which are incorporated herein by their entireties.

### REFERENCE TO SEQUENCE LISTING

The instant application contains a Sequence Listing as an XML file entitled “17424-113-US-COA_SequenceListing” created on May 28, 2026 and having a size of 129,982 bytes. The Sequence Listing is hereby incorporated by reference.

### TECHNICAL FIELD

The present disclosure belongs to the field of biopharmaceuticals, in particular to a CD3-targeting antibody, bispecific antibody and use thereof.

### BACKGROUND

T-lymphocytes are an important class of cells involved in the adaptive immune response, and T cells recognize antigens through the T cell receptor (TCR). The TCR does not recognize antigen surface epitopes directly, but specifically recognizes antigen-peptide-MHC molecular complexes (pMHC) presented on the surface of antigen-presenting cells (APCs) or target cells. The specificity of the T cell response is mediated by the recognition of pMHC by molecular complexes of the TCR and CD3. TCR is a heterodimer composed of two different transmembrane polypeptide chains with four peptide chains including α, β, γ and δ; according to the different combinations of peptide chains, TCR is divided into TCRαβ and TCRγδ. CD3 has different transmembrane polypeptide chains, i.e., γ, δ, ε, and ζ, which interact to form homodimers or heterodimers as part of the TCR-CD3 complexes. For example, the TCR-CD3 complexes include TCRαβ dimer, CD3γε dimer, CD3δε dimer, and CD3ζζdimer. Since the cytoplasmic region of the TCR peptide chain is very short, it is generally assumed that the activation signal generated by antigen recognition by the TCR is transduced into the T cell by the CD3 peptide chain.

Due to the important role of CD3 in initiating the immune response, the signal transduction targeting TCR-CD3 signaling, particularly monoclonal antibodies targeting CD3, are considered to be effective agents that can modulate the immune process and be used to treat inflammatory or autoimmune diseases. In fact, the anti-CD3 antibody Orthoclone OKT3 was the first approved therapeutic antibody. OKT3 was first approved by the US FDA in 1985 for the treatment of acute rejection after organ transplantation. Although the immunosuppressive capacity resulting from repeated administration of OKT3 provided an effective treatment for rejection after kidney transplantation, its application was limited by the first toxic dose response syndrome; the syndrome thought to be associated with OKT3-mediated T cell activation and cytokine release. Subsequently, OKT3 was withdrawn from the market in 2010 due to severe cytokine storm and immunogenicity problems associated with murine antibodies, among other factors.

Another problem with CD3 antibodies is that many CD3 antibodies have been found to be species-specific, for example, OKT3 reacts with CD3 of chimpanzee but not with CD3 of other primates, such as macaque CD3 homologs, or murine CD3 homologs. The species specificity of CD3 monoclonal antibodies is a significant barrier to their development as antibody drugs for the treatment of human diseases. Any new candidate drug must undergo rigorous preclinical validation before it can be used in clinical trials in human patients. The purpose of preclinical testing is to confirm that the candidate drug has the desired activity and, most importantly, that the candidate drug is safe. Preclinical safety testing involves the administration of the candidate drug to the species of interest, preferably Non-Human Primates. However, higher primates, particularly chimpanzees, are considered endangered species and the use of such animals for drug safety testing is highly restricted. The species described in the art suitable for safety evaluation testing may be macaques, in particular cynomolgus monkeys. However, it is difficult to provide valid preclinical safety evaluation data of CD3 antibodies that lack primate species-specific cross-reactivity. Among the known antibodies that bind to human CD3, SP34 is one of the very few that can bind to multiple primate CD3s (e.g., human and cynomolgus monkey CD3) (See, Salmeron, A. et al, J Immunol 147 (1991) 3047-3052; Conrad M. L., et. al, Cytometry A 71 (2007) 925-933).

Although monoclonal antibodies of CD3 have been clinically validated for their effectiveness in certain diseases, however, in recent years, CD3 antibodies have been more often used in the development of bispecific antibody drugs. Currently, CD3-based Bi-specific T cell engager antibodies (BsTCE) account for more than half of the bispecific antibody programs in the clinical or preclinical stage worldwide. The CD3 bispecific antibodies BsTCE, on the one hand, show the same strong efficacy as CAR-T cell therapy, and on the other hand, they can be produced and commercialized like traditional monoclonal antibodies. Among the bispecific antibodies currently approved for marketing worldwide, the earliest Catumaxomab (approved by Europe EMA in 2009, and withdrawn from the US in 2013) and Blinatumomab (approved by FDA in 2014) are both BsTCEs. CD3 antibody is an important component in the construction of BsTCE. BsTCE bispecific antibody can bind to two targets at the same time, one end recognizes Tumor-associated antigen (TAA) on the surface of tumor cells, while the other end binds to the CD3 molecule on T cells. In the presence of tumor cells, the binding of BsTCE bispecific antibody to the surface of tumor cells can recruit and activate T cells near the tumor cells, which in turn kills the tumor cells. When designing and constructing various structures of BsTCE bispecific antibodies, the selection and optimization of CD3 antibodies is of paramount importance. First, the species specificity of CD3 monoclonal antibodies is very important, especially monkey cross-reaction. Second, the affinity of the CD3 antibody to the CD3 complex is also very important; CD3 antibody with high affinity may confine the antibody to the spleen and other areas, making it difficult to contact with the tumor; and high affinity may also over stimulate T cells, resulting in high level of cytokine release. Third, CD3 antibody binding valence bonds also play an important role, it was previously found that multivalent forms of CD3 bispecific antibodies may cause side effects by activating T cells without binding tumor-associated antigens, and thus the vast majority of CD3 bispecific antibodies under investigation are in the form of monovalent CD3.

In addition to CD3 antibodies, the structural design of BsTCE bispecific antibodies is also very important. There are various structures of BsTCE bispecific antibodies, which can be divided into two main categories: IgG-like structures containing Fc and antibody fragment structures without Fc. For example, Blinatumomab is a single polypeptide chain structure consisting of two single-chain variable region antibody fragments (scFv) in series, but this structure has a short half-life, requiring continuous intravenous perfusion, and is very inconvenient to use. Fc-containing structures are used in many BsTCE bispecific antibodies therefore to improve molecular stability and pharmacokinetic properties. However, since the CD3-binding domain in BsTCE usually requires a monovalent form, Fc-containing structures are often asymmetric. There are many technical difficulties to be overcome in these asymmetric structures containing Fc, such as the heavy chain homodimerization problem in the asymmetric structure, the light chain mismatch problem, the molecular cross-linking caused by Fcγ receptor and the functional effects such as ADCC or CDC, etc. Different asymmetric structures can be chosen for the construction of BsTCE bispecific antibodies from anti-TAA IgG antibodies and anti-CD3 IgG antibodies [FIG. 16(A)], one of the commonly used structures is an IgG-like structure that retains two independent Fab domains, which contains four different polypeptide chains [two different heavy chains and two different light chains, structure shown in FIG. 16(B)], with an approximate molecular weight to that of a conventional monoclonal antibody; this structure may bring about by-products containing multiple combinations due to containing many different polypeptide chains, which poses a great challenge to the expression purification and production process of the antibody. If the Fab of the CD3 antibody is modified into a scFv structure, the “four-chain” structure can be changed into a “three-chain” structure [shown in FIG. 16(C)], further reducing the number of by-product combinations and thus the complexity of its production. In order to construct BsTCE bispecific antibody, the present inventors tried to convert SP34 mouse anti-IgG into scFv, but no matter which (VH/VL) arrangement mode was adopted or the length of the linking peptide was changed, no stable scFv could be obtained, so a stable anti-CD3 monoclonal antibody, especially its stable scFv structure, is urgently needed in the art.

In summary, there is an urgent need in the art for a CD3 antibody that is capable of binding to primate CD3, has a suitable CD3 binding capacity, and has a stable single-chain scFv structure.

### SUMMARY

The technical problem to be solved in the art is to overcome the defect of lacking low-antigenic, effective and safe anti-CD3 antibodies and bispecific antibodies with asymmetric structures, the present disclosure provides a CD3-targeting antibody, bispecific antibody and use thereof.

To solve the above-mentioned technical problem, the technical solution provided by the first aspect of the present disclosure is: a CD3-targeting antibody, comprising a light chain variable region (VL) and a heavy chain variable region (VH); wherein the amino acid sequence of the VL is set forth in SEQ ID NO: 56 or a mutant thereof, the VH is a mutant of the amino acid sequence set forth in SEQ ID NO: 42 comprising one or more mutations at positions 30, 73, 76, 78, 93 and 94 (according to Chothia numbering scheme). The mutation can cause addition, deletion or substitution of one or more amino acid residues in the original amino acid sequence. The CD3 -targeting antibody of the present disclosure alters the binding capacity to T cells and reduces the level of cytokine release, and thus is expected to reduce the toxicity associated with cytokine release syndrome.

In a preferred example, the VH has mutations at positions selected from the following groups:
(a) position 30;
(b) positions 30, 73 and 76;
(c) positions 30, 93 and 94;
(d) positions 30, 73 and 93;
(e) positions 30 and 93;
(f) positions 30, 76 and 78;
(g) positions 73, 76, 93 and 94;
(h) positions 76, 78 and 93;
(I) positions 30, 73, 76, 93 and 94;
(j) positions 30, 76, 78 and 93.

In a preferred example, the VH has mutations selected from the following groups:
(a) N30S;
(b) N30S, D73N and S76N;
(c) N30S, V93A, and R94K;
(d) N30S, D73N, and V93A;
(e) N30S and V93T;
(f) N30S, S76N and L78A;
(g) D73N, S76N, V93A, and R94K;
(h) S76N, L78A, and V93T;
(i) N30S, D73N, S76N, V93A, and R94K;
(j) N30S, S76N, L78A, and V93T.

Provided that the VH of the antibody has the above-defined mutations, the antibody of the present disclosure is further mutated on the amino acid sequence of the VL set forth in SEQ ID NO: 56, or on the amino acid sequence of the VH set forth in SEQ ID NO: 42, and the resulted amino acid sequence has 80%, 85%, 90%, 95%, 98%, 99% or more identity with the original amino acid sequence, and the amino acid sequences that maintain or improve the function of the antibody are also within the scope of protection of the present disclosure.

In a preferred example, the amino acid sequence of the VH is set forth in any one of SEQ ID NOs: 43-55, and/or, the amino acid sequence of the VL is set forth in any one of SEQ ID NOs: 57-60.

In a preferred example,
the amino acid sequence of the VH is set forth in SEQ ID NO: 44, and the amino acid sequence of the VL is set forth in SEQ ID NO: 58; or,
the amino acid sequence of the VH is set forth in SEQ ID NO: 51, and the amino acid sequence of the VL is set forth in SEQ ID NO: 58; or,
the amino acid sequence of the VH is set forth in SEQ ID NO: 44, and the amino acid sequence of the VL is set forth in SEQ ID NO: 60; or,
the amino acid sequence of the VH is set forth in SEQ ID NO: 51, and the amino acid sequence of the VL is set forth in SEQ ID NO: 60; or,
the amino acid sequence of the VH is set forth in SEQ ID NO: 45, and the amino acid sequence of the VL is set forth in SEQ ID NO: 58; or,
the amino acid sequence of the VH is set forth in SEQ ID NO: 52, and the amino acid sequence of the VL is set forth in SEQ ID NO: 58; or,
the amino acid sequence of the VH is set forth in SEQ ID NO: 43, and the amino acid sequence of the VL is set forth in SEQ ID NO: 58; or,
the amino acid sequence of the VH is set forth in SEQ ID NO: 43, and the amino acid sequence of the VL is set forth in SEQ ID NO: 60; or,
the amino acid sequence of the VH is set forth in SEQ ID NO: 50, and the amino acid sequence of the VL is set forth in SEQ ID NO: 58; or,
the amino acid sequence of the VH is set forth in SEQ ID NO: 47, and the amino acid sequence of the VL is set forth in SEQ ID NO: 58; or,
the amino acid sequence of the VH is set forth in SEQ ID NO: 48, and the amino acid sequence of the VL is set forth in SEQ ID NO: 58; or,
the amino acid sequence of the VH is set forth in SEQ ID NO: 49, and the amino acid sequence of the VL is set forth in SEQ ID NO: 58; or,
the amino acid sequence of the VH is set forth in SEQ ID NO: 53, and the amino acid sequence of the VL is set forth in SEQ ID NO: 58; or,
the amino acid sequence of the VH is set forth in SEQ ID NO: 54, and the amino acid sequence of the VL is set forth in SEQ ID NO: 58; or,
the amino acid sequence of the VH is set forth in SEQ ID NO: 43, and the amino acid sequence of the VL is set forth in SEQ ID NO: 57; or,
the amino acid sequence of the VH is set forth in SEQ ID NO: 44, and the amino acid sequence of the VL is set forth in SEQ ID NO: 57; or,
the amino acid sequence of the VH is set forth in SEQ ID NO: 43, and the amino acid sequence of the VL is set forth in SEQ ID NO: 59; or,
the amino acid sequence of the VH is set forth in SEQ ID NO: 44, and the amino acid sequence of the VL is set forth in SEQ ID NO: 59; or,
the amino acid sequence of the VH is set forth in SEQ ID NO: 51, and the amino acid sequence of the VL is set forth in SEQ ID NO: 57; or,
the amino acid sequence of the VH is set forth in SEQ ID NO: 55, and the amino acid sequence of the VL is set forth in SEQ ID NO: 58; or,
the amino acid sequence of the VH is set forth in SEQ ID NO: 46 and the amino acid sequence of the VL is set forth in SEQ ID NO: 58.

In a preferred example, the antibody comprises a single chain variable antibody (scFv) of VL-linker-VH or VH-linker-VL; preferably, the linker (i.e., linker peptide) is (GGGGS) n [abbreviation (G₄S)ₙ] or a variant thereof, wherein n is a non-zero natural number, preferably 1 to 20, more preferably the amino acid sequence of the linker is set forth in SEQ ID NO: 65, SEQ ID NO: 66, or SEQ ID NO: 67; more preferably, the amino acid sequence of the scFv is set forth in SEQ ID NO: 73, SEQ ID NO: 74, SEQ ID NO: 75, SEQ ID NO: 78, SEQ ID NO: 79 or SEQ ID NO: 80; further preferably, the antibody further comprises fragment crystallizable (Fc), the Fc linked to the scFv by a Hinge.

In a preferred example, the antibody further comprises a constant region, preferably a human constant region; preferably, the human constant region comprises a human light chain constant region and a human heavy chain constant region, and the human light chain constant region is preferably a human k light chain constant region as shown in SEQ ID NO: 61 or a human A light chain constant region as shown in SEQ ID NO: 62; more preferably, the human heavy chain constant region is hIgG1, hIgG2, hIgG3, hIgG4, or a variant thereof, preferably a heavy chain constant region as shown in SEQ ID NO: 63 or SEQ ID NO: 64.

To solve the above-mentioned technical problem, the technical solution provided by the second aspect of the present disclosure is: a bispecific antibody. The bispecific antibody of the present disclosure has a three-chain structure, which can reduce the number of by-product combinations and thus the complexity of its production; but it is not possible to develop the bispecific antibody by slightly modifying the antibody of the existing technology. As described in the background, in order to construct BsTCE bispecific antibody, the present inventors tried to convert SP34 mouse anti-IgG into scFv, but no matter which (VH/VL) arrangement mode was adopted or the length of the linker peptide was changed, no stable scFv could be obtained. After several mutation designs and validations, the inventors found that only some of these mutations could keep the scFv in a stable structure. The bispecific antibody of the present disclosure comprising a first protein functional region and a second protein functional region, wherein the first protein functional region comprises the CD3-targeting antibody of the first aspect of the present disclosure; preferably, the bispecific antibody comprises the following three chains: (1) VL1-linker-VH1-Hinge-CH2-CH3 (knob) or VH1-linker-VL1-Hinge-CH2-CH3 (knob) of the first protein functional region, (2) VH2-CH1-Hinge-CH2-CH3 (hole) of the second protein functional region, and (3) VL2-CL of the second protein functional region; the second protein functional region is a non-CD3-targeting antibody, preferably a B7H4-targeting antibody or a ROR1-targeting antibody, and the linker is preferably (G₄S) n, wherein n is a non-zero natural number, preferably 1 to 20, and more preferably the amino acid sequence of the linker is set forth in SEQ ID NO: 65, SEQ ID NO: 66, or SEQ ID NO: 67; more preferably, the bispecific antibody comprises VL1-linker-VH1-Hinge-CH2-CH3 (knob) as shown in SEQ ID NO: 88, VH2-CH1-Hinge-CH2-CH3 (hole) as shown in SEQ ID NO: 86, and VL2-CL as shown in SEQ ID NO: 83, or, VL1-linker-VH1-Hinge-CH2-CH3 (knob) as shown in SEQ ID NO: 88, VH2-CH1-Hinge-CH2-CH3 (hole) as shown in SEQ ID NO: 87, and VL2-CL as shown in SEQ ID NO: 85. The bispecific antibody of the present disclosure overcome the defect of instability of the CD3 -targeting single chain antibody arm, which is stable and has the ability to bind to T cells. The bispecific antibody containing only three chains is easily to be prepared, the production difficulty of which is reduced.

To solve the above-mentioned technical problem, the technical solution provided by the third aspect of the present disclosure is: an isolated nucleic acid, encoding the CD3-targeting antibody of the first aspect of the present disclosure or the bispecific antibody of the second aspect of the present disclosure.

To solve the above-mentioned technical problem, the technical solution provided by the forth aspect of the present disclosure is: an expression vector, comprising the isolated nucleic acid of the third aspect of the present disclosure; preferably, the expression vector is selected from a retroviral vector, a lentiviral vector, an adenovirus vector, and an adeno-associated virus vector.

To solve the above-mentioned technical problem, the technical solution provided by the fifth aspect of the present disclosure is: a genetically modified cell, transfected with the expression vector of the forth aspect of the present disclosure; preferably, the genetically modified cell is a eukaryotic cell.

To solve the above-mentioned technical problem, the technical solution provided by the sixth aspect of the present disclosure is: a pharmaceutical composition, comprising the CD3 -targeting antibody of the first aspect of the present disclosure, the bispecific antibody of the second aspect of the present disclosure, the genetically modified cell of the fifth aspect of the present disclosure, and a pharmaceutically acceptable carrier; preferably, the pharmaceutical composition further comprises an immune checkpoint antibody.

To solve the above-mentioned technical problem, the technical solution provided by the seventh aspect of the present disclosure is: a use of the CD3-targeting antibody of the first aspect of the present disclosure, the bispecific antibody of the second aspect of the present disclosure, the isolated nucleic acid of the third aspect of the present disclosure, the expression vector of the forth aspect of the present disclosure, the genetically modified cell of the fifth aspect of the present disclosure or the pharmaceutical composition of the sixth aspect of the present disclosure in the manufacture of a medicament for the treatment of tumor.

To solve the above-mentioned technical problem, the technical solution provided by the eighth aspect of the present disclosure is: a kit combination, comprising a kit A and a kit B; the kit A comprises the CD3-targeting antibody of the first aspect of the present disclosure, the bispecific antibody of the second aspect of the present disclosure, the genetically modified cell of the fifth aspect of the present disclosure or the pharmaceutical composition of the sixth aspect of the present disclosure; the kit B comprises other antibodies, bispecific antibodies, genetically modified cells or pharmaceutical compositions, the other antibodies, bispecific antibodies, genetically modified cells or pharmaceutical compositions targeting CD3, B7H4, ROR1 or other targets. The kit A and kit B can be used in any order, kit A can be used before kit B, or kit B can be used before kit A. The drug in kit A is present in an injectable form such as an injection, and the drug in kit B is present in an injectable form such as an injection, or in a swallowable form such as a tablet or pill.

The CD3-targeting antibody of the first aspect of the present disclosure, the bispecific antibody of the second aspect of the present disclosure, the genetically modified cell of the fifth aspect of the present disclosure, the pharmaceutical composition of the sixth aspect of the present disclosure or the kit combination of the eighth aspect of the present disclosure may be administered to a patient for the treatment of the relevant tumor.

On the basis of common sense in the art, the above-mentioned preferred conditions can be combined arbitrarily to obtain preferred examples of the present disclosure.

The reagents and raw materials used in the present disclosure are all commercially available.

The positive and progressive effects of the present disclosure are:
1. The monoclonal antibody of the present disclosure alters the binding capacity to T cells and reduces the level of cytokine release, and thus is expected to reduce the toxicity associated with cytokine release syndrome.
2. The bispecific antibody prepared from it overcome the defect of instability of the CD3 -targeting single chain antibody arm, which is stable and has the ability to bind to T cells.
3. The bispecific antibody containing only three chains is easily to be prepared, the production difficulty of which is reduced.

### BRIEF DESCRIPTION OF THE DRAWINGS

FIG. 1 shows the HPLC-SEC results of the CD3 single-chain antibody after one-step purification: (A) PR000275, (B) PR000276, (C) PR000307, and (D) PR000308.

FIG. 2 shows the sequence alignment of the humanized mutants of SP34 VH. The initial sequence of SP34_VH is set forth as SEQ ID NO: 42, and the variant sequences of VH3730, VH3731, VH3732, VH3733, VH3734, VH3735, VH3230, VH3231, VH3232, VH3233, VH3234, VH3235 and VH3236 are set forth as SEQ ID NOs: 50-55 and 43-49 respectively. The template sequences of IGHV3-73*01|GHJ1*01 and IGHV3-23*01|IGHJ1*01 are set forth as SEQ ID NOs: 89 and 90 respectively.

FIG. 3 shows the sequence alignment of humanized mutants of SP34 VL. The initial sequence of SP34_VL is set forth as SEQ ID NO: 56, and the variant sequences of VL7460, VL7461, VK1392 and VK1393 are set forth as SEQ ID NOs: 57-60. The template sequences of IGLV7-46*02|IGLJ2*01 and IGKV1-39*01|IGKJ4*01 are set forth as SEQ ID NOs: 91 and 92.

FIG. 4 shows the differences in significant sites of different VH/VL mutant sequences, wherein (A) shows the VH mutant sequence and (B) shows the VL mutant sequence.

FIG. 5 shows (A) SDS-PAGE results and (B) HPLC-SEC results of the CD3 single chain antibody PR000510 after one-step purification.

FIG. 6 shows the binding capacity of the CD3 antibody PR000260 to (A) recombinant CHOK1 cells overexpressing human CD3 and (B) recombinant CHOK1 cells overexpressing cynomolgus monkey CD3.

FIG. 7A to FIG. 7G show the binding capacity of the CD3 antibody to human T cells, including the binding curve and MFI relative intensity (fluorescence intensity MFI of the antibody binding to human T cells at specific concentrations, and relative ratio compared to the initial antibody PR000260 (SP34)) or MFI maximum, wherein FIG. 7A: PR000511, PR000512, PR000513, PR000514 and PR000260 bind to human T cells, FIG. 7B: PR001848, PR001849 and PR000260 bind to human T cells, FIG. 7C: PR002467, PR002468, PR002469, PR002470, PR002471, PR002472, PR001848 and PR000260 bind to human T cells, FIG. 7D: PR001848, PR002742, PR002743 and PR000260 bind to human T cells, FIG. 7E: PR002833, PR002834, PR002835, PR002836, PR002837, PR002742, PR001848, PR002469 and PR000260 bind to human T cells, FIG. 7F: PR003886, PR001848 and PR002742 bind to human T cells, FIG. 7G: PR001848, PR002469 and PR004616 bind to human T cells.

FIG. 8 shows the binding capacity of the CD3 single-chain antibody to human T cells, including the binding curve and MFI relative intensity (fluorescence intensity MFI of the antibody binding to human T cells at specific concentration, and relative ratio compared to the initial antibody PR000260 (SP34)), wherein (A) PR000510, PR000624, PR000627 and PR000260 bind to human T cells, (B) PR001850 and PR000260 bind to human T cells.

FIG. 9 shows the binding capacity of the CD3 antibody to cynomolgus monkey T cells.

FIG. 10A to FIG. 10G show the capacity of the CD3 antibody to activate human T cells to produce cytokine IFN-γ, wherein FIG. 10A: PR000511, PR000512, PR000513, PR000514 and PR000260 activate T cells, FIG. 10B: PR001848, PR001849 and PR000260 activate T cells, FIG. 10C: PR002468, PR002469, PR002471 and PR001848 activate T cells, FIG. 10D: PR002742, PR001848 and PR000260 activate T cells, FIG. 10E: PR002833, PR002834, PR002835,
PR002836, PR002837 and PR000260 activate T cells, FIG. 10F: PR003886, PR001848 and PR002742 activate T cells, FIG. 10G: PR001848, PR002469 and PR004616 activate T cells.

FIG. 11 shows the capacity of the CD3 single-chain antibody (PR000510, PR000623, PR000624, PR000627 and PR000260) to activate human T cells to produce cytokine IFN-γ.

FIG. 12 shows the SDS-PAGE results of samples of the bispecific antibody (A) PR002883 and (B) PR002885 obtained one-step purification.

FIG. 13 shows the binding capacity of the monoclonal antibody and the bispecific antibody to (A) SK-BR-3 cells and (B) human T cells.

FIG. 14 shows the target cell killing capacity mediated by the bispecific antibody PR002883 in vitro; wherein, (A) shows SK-BR-3 cell killing and (B) shows IFN-γ release levels.

FIG. 15 shows the binding capacity of the monoclonal antibody and the bispecific antibody to (A) Panc-1 cells and (B) human T cells.

FIG. 16 shows the structure of the monoclonal antibody or the bispecific antibody; (A) IgG structure, (B) asymmetric “four chain” structure, (C) asymmetric “three chain” structure containing the single chain antibody.

### DETAILED DESCRIPTION OF THE PREFERRED EMBODIMENT

The present disclosure is further illustrated below by way of examples, but the disclosure is not thereby limited to the scope of the described examples. The experimental methods for which specific conditions are not indicated in the following examples were selected according to the conventional methods and conditions, or according to the commodity specification.

In this application, the term “antibody” generally refers to a protein comprising a moiety that binds to an antigen and optionally allows the moiety that binds to the antigen to adopt a scaffold or skeleton moiety of the conformation that promotes binding of the antibody to the antigen. The antibody typically may comprise an antibody light chain variable region (VL), an antibody heavy chain variable region (VH), or both. The VH and VL regions may be further divided into hypervariable regions called complementarity determining regions (CDR), which are scattered in more conservative regions called framework regions (FR). Each of the VH and VL may consist of three CDR and four FR regions, which may be arranged in the following order from the amino end to the carboxyl end: FR1, CDR1, FR2, CDR2, FR3, CDR3, and FR4. The variable regions of the heavy and light chains contain binding domains that interact with the antigen.

Examples of the antibody include, but are not limited to, antibody, antigen-binding fragment (Fab, Fab′, F(ab)2, Fv fragment, F(ab′)2, scFv, di-scFv and/or dAb), immunoconjugate, multispecific antibody (e.g., bispecific antibody), antibody fragment, antibody derivative, antibody analog or fusion protein, and the like, as long as they show the desired antigen-binding activity.

In this application, the term “variable” generally refers to the fact that certain parts of the sequence of the variable domain of the antibody differ substantially, which forms the binding and specificity of various specific antibodies to their particular antigen. However, the variability is not uniformly distributed throughout the variable region of the antibody. It is concentrated in three segments of the light and heavy chain variable regions, known as the complementary determining region (CDR) or high variability region (HVR). The more highly conserved part of the variable domain is known as the framework (FR). The variable structure domains of the natural heavy and light chains each contain four FR regions, most of which adopt the β-fold conformation and are connected by three CDRs that form a loop linkage and, in some cases, form part of the β-fold structure. The CDRs in each chain are in close proximity to each other through the FR region and form the antigen binding site of the antibody together with the CDR from the other chain, the constant regions are not directly involved in antibody-antigen binding, but exhibit different effector functions, such as involvement in antibody-dependent cytotoxicity of the antibody. In the present art, the CDR of an antibody can be defined by a variety of methods, such as the Kabat definition scheme based on sequence variability (see, Kabat et al., Sequences of proteins of immunological interest, Fifth Edition, National Institutes of Health, Bethesda, Maryland (1991)) and the Chothia definition scheme based on the location of structural loop regions (see, Al-Lazikani et al., JMol Biol 273:927-48, 1997). In the present application, the Combined definition scheme comprising the Kabat definition and the Chothia definition is also used to identify amino acid residues in variable domain sequences and full-length antibody sequences (Table 1).

TABLE 1
The CDR definition rules of antibodies in the present
application (see http://bioinf.org.uk/abs/)

| CDR region | Rabat definition | Chothia definition | Combined definition |
|---|---|---|---|
| LCDR1 | L24--L34 | L24--L34 | L24--L34 |
| LCDR2 | L50--L56 | L50--L56 | L50--L56 |
| LCDR3 | L89--L97 | L89--L97 | L89--L97 |
| HCDR1 | H31--H35 | H26--H32 | H26--H35 |
| HCDR2 | H50--H65 | H52--H56 | H50--H65 |
| HCDR3 | H95--H102 | H95--H102 | H95--H102 |

Wherein, Laa-Lbb may refer to the amino acid sequence from position aa (Chothia numbering scheme) to position bb (Chothia numbering scheme) starting from the N-terminal of the antibody light chain; Haa-Hbb may refer to the amino acid sequence from position aa (Chothia coding scheme) to position bb (Chothia numbering scheme) starting from the N-terminal of the antibody heavy chain. For example, L24-L34 may refer to the amino acid sequence from position 24 to position 34 starting from the N-terminal of the light chain of the antibody according to the Chothia numbering scheme; H26-H32 may refer to the amino acid sequence from position 26 to position 32 starting from the N-terminal of the heavy chain of the antibody according to the Chothia numbering scheme.

The antibody Fc domain-mediated effector functions such as ADCC and CDC also have very important biological functions, different IgG isoforms have different ADCC or CDC functions, for example, IgG1 and IgG3 have strong ADCC and CDC effects, while IgG2 and IgG4 have relatively weak effects. In addition, the original effector function of Fc can also be modulated by amino acid mutations or modifications to alter the binding ability of Fc to the Fc receptor. For example, the “LALA” double mutation (L234A/L235A) in IgG1 significantly reduces the affinity to FcγRIIIA (CD16A) and thus reduces the ADCC effect. In addition, the P329G mutation significantly reduces the binding of IgG1 multiple Fcγ receptors (see, Schlothauer T, Herter S, Koller C F, et al. Protein Eng Des Sel. 2016 Oct.; 29 (10): 457-466). In this application, in order to reduce the binding of CD3 antibodies to the Fcγ receptor, “LALA” double mutation (L234A/L235A) or the “LALAPG” triple mutation (L234A/L235A/P329G) was introduced into the Fc of these CD3 antibodies.

### Example 1 Preparation and Characterization Analysis of Recombinant Antibodies 1.1 Preparation of IgG Recombinant Antibodies

After obtaining the light and heavy chain variable domain sequences encoding the antibody molecule, the recombinant antibody molecule can be prepared by fusion expression of the light and heavy chain variable domain sequences with the corresponding human antibody light and heavy chain constant domain sequences using conventional recombinant DNA techniques. In this example, the antibody heavy chain variable domain sequence (VH) is genetically synthesized and cloned into the mammalian cell expression plasmid vector encoding the human IgG1 antibody heavy chain constant domain sequence to obtain the full-length heavy chain of IgG1 antibody by encoding, and the “LALA” double mutation (L234A/L234A) (SEQ ID NO: 63) or the “LALAPG” triple mutation (L234A/L235A/P329G) (SEQ ID NO: 64) is introduced in the IgG1 heavy chain constant region to reduce the antibody binding to the Fcγ receptor. The sequence of antibody light chain variable domain (VL) is genetically synthesized and cloned into the mammalian cell expression plasmid vector encoding the sequence of human antibody k light chain constant domain (SEQ ID NO: 61) to obtain the full length k light chain of antibody by encoding; alternatively, VL is genetically synthesized and cloned into the mammalian cell expression plasmid vector encoding the sequence of human antibody 2 light chain constant domain (SEQ ID NO: 62) to obtain the full length 2 light chain to produce antibody by encoding.

By co-transfecting mammalian host cell (e.g., human embryonic kidney cell HEK293) with plasmids encoding the antibody heavy chain and plasmids encoding the antibody light chain, the purified recombinant antibody with correctly paired assembly of light and heavy chains can be obtained using conventional recombinant protein expression and purification techniques. Specifically, the HEK293 cells were expanded in medium FreeStyle™ F17 Expression Medium (Thermo, #A1383504). Before transient transfection, adjusted the cell concentration to 6-8×10⁵ cells/ml and incubated them in shaker at 37° C. 8% CO₂ for 24 hours at the cell concentration of 1.2×10⁶ cells/ml. 30 ml of cultured cells were prepared. The plasmid encoding the heavy chain and the plasmid encoding the light chain were mixed in a ratio of 2:3, a total of 30 μg plasmid were dissolved in 1.5 mL Opti-MEM reduced serum medium (Thermo, #31985088) and filtered through the 0.22 μm membrane. Then 1.5 mL opti-MEM was dissolved into 120 μL 1 mg/mL PEI (Polysciences, #23966-2), and left standing for 5 minutes. PEI was slowly added to the plasmid, thereafter incubating for 10 minutes at room temperature. The mixed solution of plasmid PEI was slowly added into a culture flask dropwise while shaking the culture flask. The transfected cells were incubated at 37° C., 8% CO₂ in a shaker for 5 days. The cell viability were observed after 5 days. Then cultures were harvested by centrifugation at 3300g for 10 minutes to collect the supernatant. Impurities in the supernatant was removed by centrifugation at high speed. The gravity column (Bio-Rad, #7311550) containing MabSelect™ (GE Healthcare Life Science, #71-5020-91 AE) was equilibrated with PBS (pH 7.4) and rinsed with 2-5 times of the column volume of PBS. The column was loaded with the supernatant sample and rinsed with 5-10 times of the column volume of PBS. Then the target protein was elutd with 0.1 M glycine at pH 3.5, later adjusted to neutral pH with Tris-HCl at pH 8.0, finally concentrated using the ultrafiltration tube (Millipore, #UFC901024) and exchanged to PBS buffer and to obtain the purified recombinant antibody solution. At last, measured the concentration by NanoDrop (Thermo Scientific™ NanoDrop™ One), dispensed and stored the purified recombinant antibody solution for backup.

1.2 Preparation of Monovalent scFv-his Recombinant Antibodies

The VH and VL sequences of the antibody were linked by a flexible peptide (linker) to obtain a single polypeptide chain encoding both VH and VL, i.e., a single chain antibody variable region fragment (scFv). If a linker peptide of suitable length, such as (G₄S) 3 (SEQ ID NO: 65) or (G₄S) 4 (SEQ ID NO: 66), is selected, VH and VL can be correctly folded and assembled into functional antibodies. Different scFv structures (VH-linker-VL or VL-linker-VH) can be constructed depending on the different arrangements of VH and VL and difference of the linker peptide. A single scFv contains an antigen-binding region consisting of a pair of VH and VL, which usually binds only one antigen molecule and is thus called a monovalent binding molecule.

To facilitate purification, in this example, The C-terminal of the scFv was fused with His tag composed of 6-Histidine. The plasmid encoding the scFv and His tag was genetically synthesized and cloned into expression plasmid vector for mammalian cells to obtain the plasmid encoding scFv-his, which was transfected into mammalian host cell (e.g., human embryonic kidney cell HEK293), and then the recombinant protein can be purified using conventional recombinant protein expression and purification techniques. Specifically, the HEK293 cells were expanded in medium FreeStyle™ F17 Expression Medium (Thermo, #A1383504). Before transient transfection, adjusted the cell concentration to 6-8×10⁵ cells/ml and incubated them in shaker at 37° C. 8% CO₂ for 24 hours at the cell concentration of 1.2×10⁶ cells/ml. 30 ml of cultured cells were prepared. 30 μg of the plasmid were dissolved in 1.5 mL Opti-MEM reduced serum medium (Thermo, #31985088) and filtered through the 0.22 μm membrane. Then 1.5 mL opti-MEM was dissolved into 120 μL 1 mg/mL PEI (Polysciences, #23966-2), and left standing for 5 minutes. PEI was slowly added to the plasmid, thereafter incubating for 10 minutes at room temperature. The mixed solution of plasmid-PEI was slowly added into a culture flask dropwise while shaking the culture flask. The transfected cells were incubated at 37° C., 8% CO₂ in a shaker for 5 days. The cell viability were observed after 5 days. Then cultures were harvested by centrifugation at 3300g for 10 minutes to collect the supernatant. Impurities in the supernatant was removed by centrifugation at high speed. The gravity column (Bio-Rad, #7311550) containing Ni Sepharose excel (GE Healthcare Life Science, #17-3712-01) Ni Sepharose excel (GE Healthcare Life Science, #17-3712-01) was equilibrated with PBS (pH 7.4) and rinsed with 2-5 times of the column volume of PBS. The column was loaded with the supernatant sample; and rinsed with 5-10 times of the column volume of PBS. Non-specifically adsorbed heteroproteins was eluted with buffer A (containing 20 mM imidazole, 150 mM phosphate, pH 8.0) first, and then the target protein with buffer B (containing 500 mM imidazole, 150 mM phosphate, pH 8.0), finally using the ultrafiltration tube (Millipore, #UFC901024) to concentrate and exchange the solution to PBS buffer and to obtain the purified recombinant antibody solution. At last, measured the concentration by NanoDrop (Thermo Scientific™ NanoDrop™ One), dispensed and stored the purified recombinant antibody solution for backup.

1.3 Preparation of Bivalent scFv-Fc Recombinant Antibodies

In this example, the scFv-Fc recombinant molecule was constructed by fusing the sequence of human IgG1 constant region Fc (Glu216-Lys447, containing the hinge region, CH2 domain and CH3 domain) at the C-terminus of scFv, and through homodimerization of Fc, a bivalent scFv-Fc dimer molecule was formed, which is capable of binding two antigen molecules simultaneously. And the “LALA” double mutation (L234A/L235A) or the “LALAPG” triple mutation (L234A/L235A/P329G) was introduced into Fc to reduce the binding of the antibody to the Fcγ receptor. The polypeptide sequence encoding scFv-Fc was genetically synthesized and cloned into the expression plasmid vector for mammalian cells to obtain the plasmid encoding scFv-Fc, which was thereafter transfected into mammalian host cells (e.g., human embryonic kidney cell HEK293), and then using the protein expression purification method described in the Example 1.1 to obtain the purified recombinant protein.

### 1.4 Protein Purity Analysis by HPLC-SEC

The purity and aggregate form of protein samples were analyzed by molecular size exclusion chromatography (SEC). The analytical column TSKgel G3000SWxl (Tosoh Bioscience, #08541, 5 μm, 7.8 mm×30 cm) was connected to the high pressure liquid chromatograph (HPLC) (Agilent Technologies, Agilent 1260 Infinity II) and equilibrated with PBS buffer at room temperature for at least 1 hour. An appropriate amount of protein sample (at least 10 μg) filtered through the 0.22 μm membrane were injected into the system, and the HPLC program was set: the column was loaded by the sample with PBS buffer at a flow rate of 1.0 ml/min for a maximum of 20 minutes. HPLC will generate Analytical reports of HPLC would be generated, which reports the retention time of the different molecular size components in the sample.

### Example 2 Recombinant Expression of Murine-Human Chimeric Antibody of CD3 Antibody SP34

SP34 is a murine-derived anti-human CD3& antibody that binds a variety of primate CD3 and functions to activate T cells. The sequences of variable region VH and VL of SP34 have been disclosed in WO2016071004A1. In this application, the amino acid sequence of VH of SP34 is set for in SEQ ID NO: 42, and its corresponding murine germline V gene is IGHV10-1; the amino acid sequence of VL of SP34 is set forth in SEQ ID NO: 56, and its corresponding murine germline V gene is IGLV1. In this example, the VH sequence of SP34 was fused with the human IgG1 antibody heavy chain constant domain sequence (SEQ ID NO: 63) comprising a “LALA” double mutation (L234A/L235A) to produce the full-length heavy chain of SP34 murine-human chimeric IgG1 antibody; the amino acid sequence of VL of SP34 was fused with the human antibody A light chain constant domain sequence (SEQ ID NO: 62) to produce the full length 2 light chain of the SP34 murine-human chimeric antibody.

The SP34 murine-human chimeric recombinant antibody PR000260 was prepared according to the method of the Example 1.1. The following Table 2 shows the data of recombinant expression of PR000260.

TABLE 2
Expression and purification of recombinant antibody PR000260

| | Expression | | | HPLC-SEC |
|---|---|---|---|---|
| Antibody | system | Purification | Yield | (monomer |
| number | (volume) | method | (mg/L) | purity %) |
| PR000260 | HEK293 (100 ml) | MabSelect | 19.30 | 99.75% |

### Example 3 Convert Murine CD3 SP34 Antibody to Recombinant scFv Antibody

The VH sequence (SEQ ID NO: 42) and VL sequence (SEQ ID NO: 56) of SP34 were linked by the flexible peptide (linker) to obtain the single polypeptide chain encoding both VH and VL, i.e., the single chain antibody variable region fragment (scFv). Depending on the different arrangements of VH and VL and different lengths of the linker peptides (SEQ ID NO: 65, SEQ ID NO: 66), different scFv structures can be constructed, and a His tag consisting of a 6-Histidine was fused at the C-terminus of the scFv for purification. The linker peptide as shown in SEQ ID NO: 67 can also be used for the construction of scFv in this application.

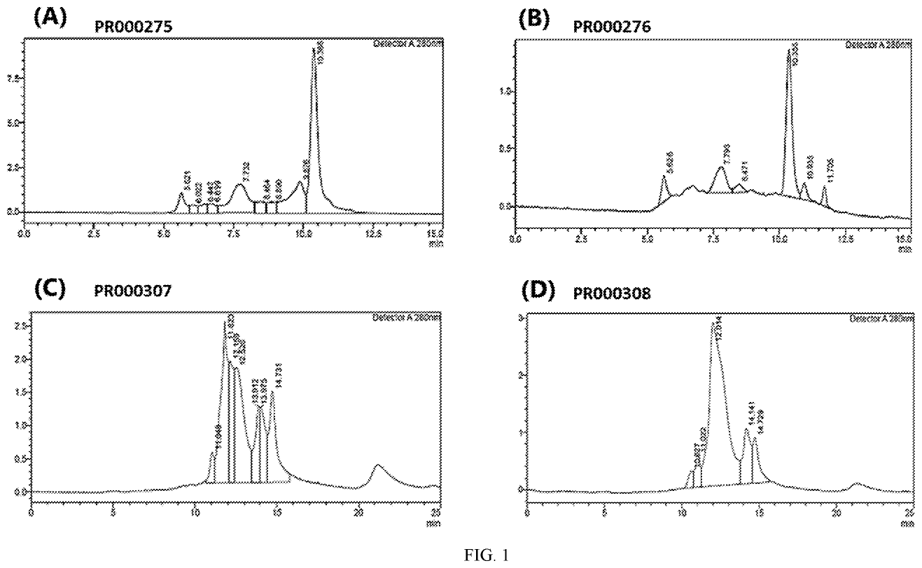

In this Example, four recombinant scFv antibody molecules (PR000275, PR000276, PR000307, PR000308) were prepared according to the method of the Example 1.2. The sequence numbers of these four recombinant scFv antibody molecules are listed in Table 3 below; the following Table 4 shows the expression data of of these four recombinant molecules; and the HPLC-SEC results of these four molecules after one-step purification are shown in FIG. 1, wherein (A) shows the result of PR000275, (B) shows the result of PR000276, (C) shows the result of PR000307, and (D) shows the result of PR000308. It can be seen that using the sequences of VH and VL of SP34 to construct scFv, no matter which (VH/VL) alignment pattern was used or the length of the linking peptide was changed, no stable scFv could be obtained.

TABLE 3
Structure and sequence number of the four recombinant antibodies

| | | | | | | | Linker | Full length |
|---|---|---|---|---|---|---|---|---|
| Antibody | VH | VL | Molecular | scFv | VH | VL | peptide | sequence |
| number | Variant | Variant | structure | Structure | (SEQ ID NOs:) | | | |
| PR000275 | SP34_VH | SP34_VL | scFv-his | VL-linker-VH | 42 | 56 | 65 | 68 |
| PR000276 | SP34_VH | SP34_VL | scFv-his | VH-linker-VL | 42 | 56 | 65 | 69 |
| PR000307 | SP34_VH | SP34_VL | scFv-his | VL-linker-VH | 42 | 56 | 66 | 70 |
| PR000308 | SP34_VH | SP34_VL | scFv-his | VH-linker-VL | 42 | 56 | 66 | 71 |

TABLE 4
Expression and purification of recombinant scFv antibodies

| | | HEK293 | Puri- | | HPLC-SEC |
|---|---|---|---|---|---|
| Antibody | | Expression | fication | Yield | (monomer |
| number | Structure | Volume | method | (mg/L) | purity %) |
| PR000275 | VL-(G4S)3-VH | 100 ml | Nickel | 2.60 | 46.61% |
| PR000276 | VH-(G4S)3-VL | 100 ml | Nickel | 0.26 | Weak signal |
| PR000307 | VL-(G4S)4-VH | 30 ml | Nickel | 3.33 | Weak signal |
| PR000308 | VH-(G4S)4-VL | 30 ml | Nickel | 3.67 | Weak signal |

### Example 4 Sequence Optimization of SP34

### 4.1 Humanization of Variable Region Sequences and Mutation of Frame Region

The “CDR transplantation” method is used for sequence humanization in this example, i.e., transplantation of the CDR of the murine antibody VH to the frame region of the human antibody VH, and transplantation of the CDR of the murine antibody VL to the frame region of the human antibody VL. The sequence of the frame region of human antibody VH or VL can be derived from human germline gene sequences or antibody sequences that have been rearranged by V(D)J or the consensus sequences of the specific VH or VL gene family of the human antibody. In this example, the frame region sequences provided by human germline gene sequences are used as humanized template sequences, i.e., the human germline V gene fragment provides the sequences of the frame regions FR1, FR2, and FR3, and the human germline J gene fragment provides the sequence of the frame region FR4. Finally, the sequences of humanized variable region (VH or VL) were constructed in the arrangement of (human) FR1-(murine) CDR1-(human) FR2-(murine) CDR2-(human) FR3-(murine) CDR3-(human) FR4.

In this example, the sequence of the human germline V gene fragment IGHV3-73*01 or the human germline V gene fragment IGHV3-23*01 was used as the humanized template in combination with the sequence of the human germline J gene fragment IGHJ1*01 to provide the frame region sequence. Amino acid mutations at one or more sites were introduced in at the position 30, position 73, position 76, position 78, position 93 or position 94 (according to Chothia numbering rules) to obtain several different VH mutant sequences.

In this example, the sequence of the human germline V gene fragment IGLV7-46*02 combined with the sequence of the human germline J gene fragment IGLJ2*01 or the sequence of the human germline V gene fragment IGKV1-39*01 combined with the sequence of the human germline J gene fragment IGKJ4*01 was used as the humanized template to provide the frame region sequence. Amino acid mutations at zero or more sites were introduced in at the position 2, position 36, position 46, position 49, position 66, position 69, position 71 or position 87 (according to Chothia numbering scheme) to obtain several different VL mutant sequences.

The following Table 5 lists the sequence numbers of the antibody variable region, its optimized mutant sequences (FV) and the sequences of the CDR and FR regions defined by CHOTHIA.

TABLE 5
Variable region of SP34 antibody and optimized
mutant sequences (FV) thereof and the sequence
list of CDR and FR regions defined by CHOTHIA

| ID | FV | FR1 | CDR1 | FR2 | CDR2 | FR3 | CDR3 | FR4 |
|---|---|---|---|---|---|---|---|---|
| SP34_VH | 42 | 5 | 1 | 8 | 3 | 11 | 4 | 21 |
| VH3730 | 50 | 7 | 1 | 10 | 3 | 17 | 4 | 22 |
| VH3731 | 51 | 7 | 1 | 10 | 3 | 18 | 4 | 22 |
| VH3732 | 52 | 7 | 2 | 10 | 3 | 18 | 4 | 22 |
| VH3733 | 53 | 7 | 2 | 10 | 3 | 19 | 4 | 22 |
| VH3734 | 54 | 7 | 2 | 10 | 3 | 20 | 4 | 22 |
| VH3735 | 55 | 7 | 2 | 10 | 3 | 17 | 4 | 22 |
| VH3230 | 43 | 6 | 1 | 9 | 3 | 12 | 4 | 22 |
| VH3231 | 44 | 6 | 1 | 9 | 3 | 13 | 4 | 22 |
| VH3232 | 45 | 6 | 2 | 9 | 3 | 13 | 4 | 22 |
| VH3233 | 46 | 6 | 2 | 9 | 3 | 12 | 4 | 22 |
| VH3234 | 47 | 6 | 2 | 9 | 3 | 14 | 4 | 22 |
| VH3235 | 48 | 6 | 2 | 9 | 3 | 15 | 4 | 22 |
| VH3236 | 49 | 6 | 2 | 9 | 3 | 16 | 4 | 22 |
| SP34_VL | 56 | 26 | 23 | 30 | 24 | 35 | 25 | 40 |
| VL7460 | 57 | 27 | 23 | 33 | 24 | 38 | 25 | 40 |
| VL7461 | 58 | 27 | 23 | 34 | 24 | 39 | 25 | 40 |
| VK1392 | 59 | 28 | 23 | 31 | 24 | 36 | 25 | 41 |
| VK1393 | 60 | 29 | 23 | 32 | 24 | 37 | 25 | 41 |

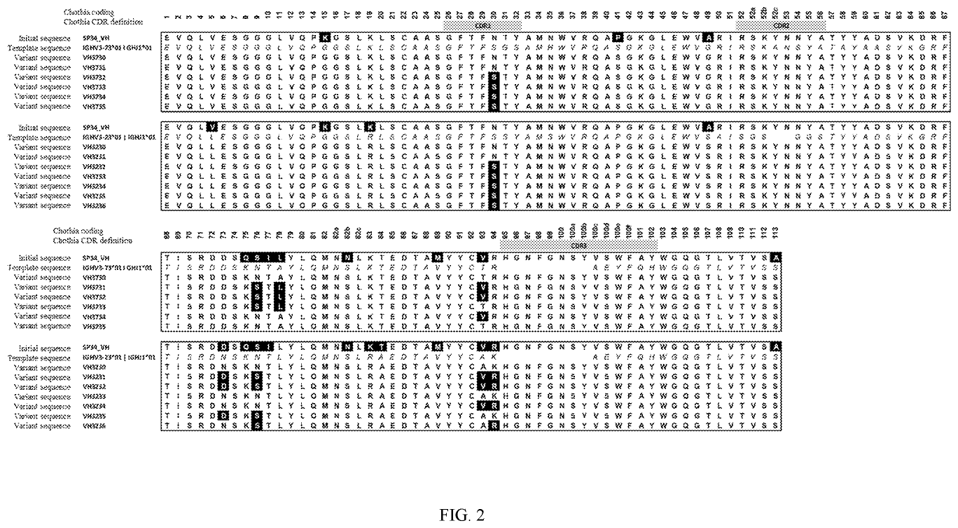

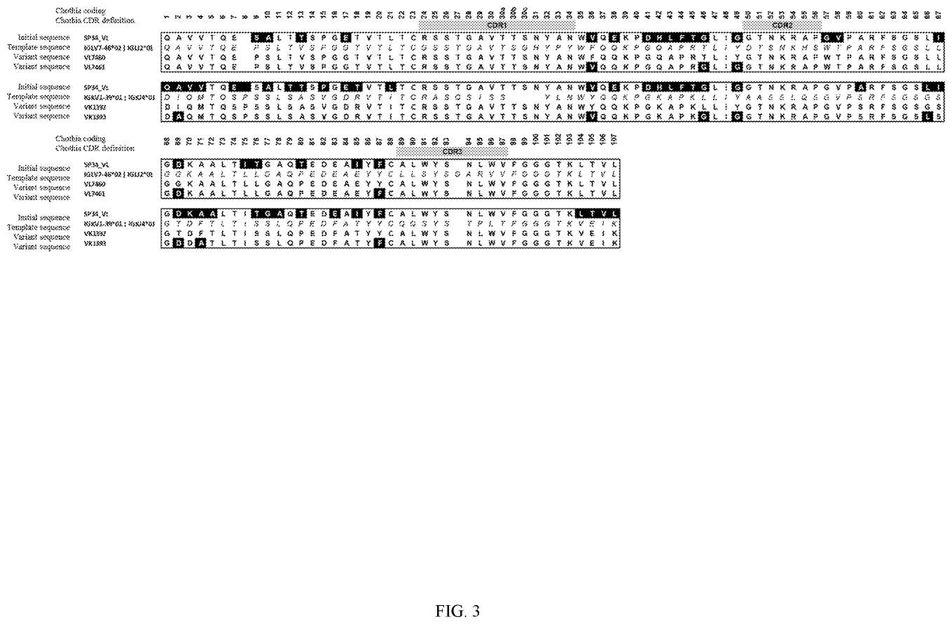

FIG. 2 lists the comparison of VH mutant sequences. FIG. 3 lists the comparison of VL mutant sequences. The differences in the sequences of VH mutants and VL mutants at significant sites are listed in (A) and (B) of FIG. 4, respectively. As can be seen from FIG. 2 to FIG. 4, the mutations of the antibody of the present disclosure on VH occurred at one or more sites selected from position 30, position 73, position 76, position 78, position 93 or position 94 of the amino acid sequence as shown in SEQ ID NO: 42. The mutations on the VL occurred at position 2, position 36, position 46, position 49, position 66, position 69, position 71 and/or position 87 of the sequence as shown in SEQ ID NO: 56. More detailed mutation information can be found in Table 5 for sequence specifics of VH3730, VH3731, VH3732, VH3733, VH3734, VH3735, VH3230, VH3231, VH3232, VH3233, VH3234, VH3235, VH3236, VL7460, VL7461, VK1392, and VK1393.

4.2 Recombinant Antibodies of Mutants with Optimized Sequences

The sequences of VH mutants and VL mutants obtained in the Example 4.1 were paired and combined, and the IgG recombinant antibody constructed according to the method in the Example 1.1, wherein the “LALA” double mutation or the “LALAPG” triple mutation was introduced into the constant region of the IgG1 heavy chain to reduce the Fc effector function. Table 6 lists the sequences of recombinant antibody molecules that have been sequence optimized. Table 7 lists the expression data of the recombinant antibodies. Except for the three IgG molecules constructed with the VH mutant VH3230, which have very low expression yields, all other IgG molecules have reasonable expression yields.

TABLE 6
Sequence list of the SP34 chimeric antibodies
or antibodies with optimized sequence

| | | | | | Heavy | Light |
|---|---|---|---|---|---|---|
| | | | | | chain | chain |
| | | | | | constant | constant |
| Antibody | VH | VL | VH | VL | region | region |
| number | Mutant | Mutant | (SEQ ID NOs:) | | | |
| PR000260 | SP34_VH | SP34_VL | 42 | 56 | 63 | 62 |
| PR000511 | VH3231 | VL7461 | 44 | 58 | 63 | 62 |
| PR000512 | VH3731 | VL7461 | 51 | 58 | 63 | 62 |
| PR000513 | VH3231 | VK1393 | 44 | 60 | 63 | 61 |
| PR000514 | VH3731 | VK1393 | 51 | 60 | 63 | 61 |
| PR001848 | VH3232 | VL7461 | 45 | 58 | 64 | 62 |
| PR001849 | VH3732 | VL7461 | 52 | 58 | 64 | 62 |
| PR002467 | VH3230 | VL7460 | 43 | 57 | 63 | 62 |
| PR002468 | VH3231 | VL7460 | 44 | 57 | 63 | 62 |
| PR002469 | VH3230 | VL7461 | 43 | 58 | 63 | 62 |
| PR002470 | VH3230 | VK1392 | 43 | 59 | 63 | 61 |
| PR002471 | VH3231 | VK1392 | 44 | 59 | 63 | 61 |
| PR002472 | VH3230 | VK1393 | 43 | 60 | 63 | 61 |
| PR002742 | VH3730 | VL7461 | 50 | 58 | 63 | 62 |
| PR002743 | VH3731 | VL7460 | 51 | 57 | 63 | 62 |
| PR002833 | VH3234 | VL7461 | 47 | 58 | 64 | 62 |
| PR002834 | VH3235 | VL7461 | 48 | 58 | 64 | 62 |
| PR002835 | VH3236 | VL7461 | 49 | 58 | 64 | 62 |
| PR002836 | VH3733 | VL7461 | 53 | 58 | 64 | 62 |
| PR002837 | VH3734 | VL7461 | 54 | 58 | 64 | 62 |
| PR003886 | VH3735 | VL7461 | 55 | 58 | 63 | 62 |
| PR004616 | VH3233 | VL7461 | 46 | 58 | 63 | 62 |

TABLE 7
Expression yield and purity of the recombinant antibodies

| | | | Yield of | HPLC-SEC |
|---|---|---|---|---|
| Antibody | | | HEK293 | monomer |
| number | VH Mutant | VL Mutant | (mg/L) | purity (%) |
| PR000260 | SP34_VH | SP34_VL | 64.33 | 99.75% |
| PR000511 | VH3231 | VL7461 | 48.33 | 99.88% |
| PR000512 | VH3731 | VL7461 | 150.00 | 100.00% |
| PR000513 | VH3231 | VK1393 | 45.00 | 100.00% |
| PR000514 | VH3731 | VK1393 | 48.33 | 100.00% |
| PR001848 | VH3232 | VL7461 | 38.50 | 99.03% |
| PR001849 | VH3732 | VL7461 | 43.50 | 99.10% |
| PR002467 | VH3230 | VL7460 | 0.60 | n/a |
| PR002468 | VH3231 | VL7460 | 107.25 | 98.84% |
| PR002469 | VH3230 | VL7461 | 10.20 | 96.85% |
| PR002470 | VH3230 | VK1392 | 0.60 | n/a |
| PR002471 | VH3231 | VK1392 | 62.50 | 94.96% |
| PR002472 | VH3230 | VK1393 | 0.30 | n/a |
| PR002742 | VH3730 | VL7461 | 14.25 | 100.00% |
| PR002743 | VH3731 | VL7460 | 44.25 | 100.00% |
| PR002833 | VH3234 | VL7461 | 18.00 | 98.92% |
| PR002834 | VH3235 | VL7461 | 24.60 | 95.88% |
| PR002835 | VH3236 | VL7461 | 13.50 | 95.58% |
| PR002836 | VH3733 | VL7461 | 26.25 | 94.68% |
| PR002837 | VH3734 | VL7461 | 17.20 | 97.36% |
| PR003886 | VH3735 | VL7461 | 116 | 98.49% |
| PR004616 | VH3233 | VL7461 | 22 | n/a |

4.3 Recombinant scFv Molecules Containing Mutants with Optimized Sequences

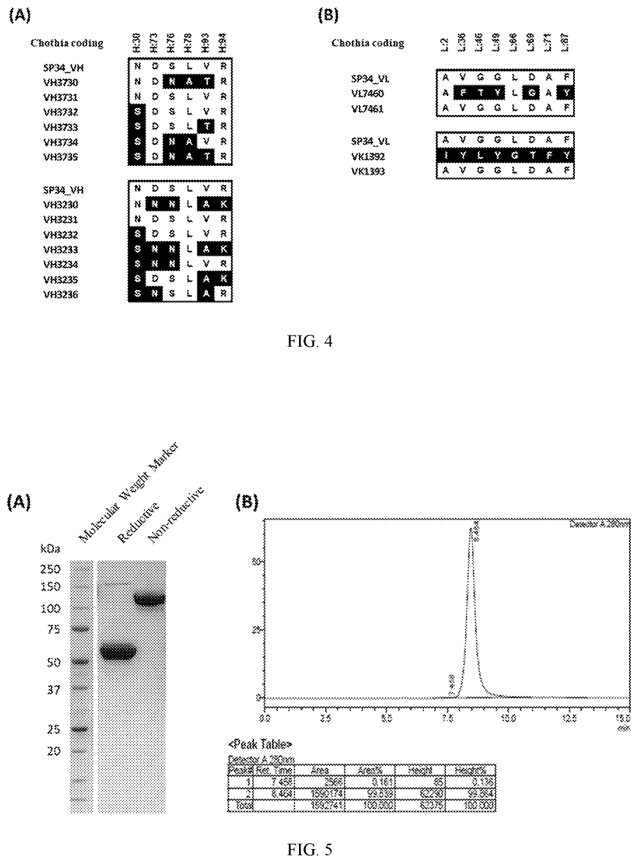

The sequences of VH mutants and VL mutants obtained in the Example 4.1 were paired and combined, and a plurality of recombinant bivalent scFv antibody molecules were obtained according to the method in the Example 1.3. The following Table 8, 9 respectively lists the sequence information and protein expression of the scFv. As can be seen in Table 9, PR000510 and PR000627 are especially better expressed and stable molecules. FIG. 5 shows the results of (A) SDS-PAGE and (B) HPLC-SEC of PR000510, which can be seen that it has a good monomeric purity with no obvious aggregates.

TABLE 8
Structure and sequence information of the scFv molecules
constructed based on the mutants with optimized sequences

| | | | | | | | Linker | Full length |
|---|---|---|---|---|---|---|---|---|
| Antibody | VH | VL | Molecular | scFv | VH | VL | peptide | sequence |
| number | Mutant | Mutant | structure | Structure | (SEQ ID NOs:) | | | |
| PR000509 | VH3731 | VL7461 | scFv-Fc | VH-linker-VL | 51 | 58 | 67 | 72 |
| PR000510 | VH3731 | VL7461 | scFv-Fc | VL-linker-VH | 51 | 58 | 67 | 73 |
| PR000623 | VH3231 | VK1393 | scFv-Fc | VH-linker-VL | 44 | 60 | 67 | 74 |
| PR000624 | VH3731 | VK1393 | scFv-Fc | VH-linker-VL | 51 | 60 | 67 | 75 |
| PR000625 | VH3231 | VK1393 | scFv-Fc | VL-linker-VH | 44 | 60 | 67 | 76 |
| PR000626 | VH3731 | VK1393 | scFv-Fc | VL-linker-VH | 51 | 60 | 67 | 77 |
| PR000627 | VH3731 | VL7461 | scFv-Fc | VL-linker-VH | 51 | 58 | 67 | 78 |
| PR000914 | VH3231 | VL7461 | scFv-Fc | VH-linker-VL | 44 | 58 | 67 | 79 |
| PR000915 | VH3231 | VL7461 | scFv-Fc | VL-linker-VH | 44 | 58 | 67 | 80 |
| PR001850 | VH3732 | VL7461 | scFv-Fc | VL-linker-VH | 52 | 58 | 67 | 81 |

TABLE 9
Expression data of the scFv antibody after sequence optimization

| | | | Yield of | HPLC-SEC |
|---|---|---|---|---|
| Antibody | | | HEK293 | monomer |
| number | VH Mutant | VL Mutant | (mg/L) | purity (%) |
| PR000509 | VH3731 | VL7461 | 0.00 | n/a |
| PR000510 | VH3731 | VL7461 | 7.00 | 99.84% |
| PR000623 | VH3231 | VK1393 | 0.60 | 93.29% |
| PR000624 | VH3731 | VK1393 | 1.80 | 100.00% |
| PR000625 | VH3231 | VK1393 | 0.00 | n/a |
| PR000626 | VH3731 | VK1393 | 0.00 | n/a |
| PR000627 | VH3731 | VL7461 | 6.00 | 100.00% |
| PR000914 | VH3231 | VL7461 | 3.33 | 78.22% |
| PR000915 | VH3231 | VL7461 | 2.67 | 86.14% |
| PR001850 | VH3732 | VL7461 | 4.88 | 50.76% |

### Example 5 Determination of the Binding Capacity of CD3 Antibody to CD3 Expressing Cells by FACs

The flow cytometryFACS was used to analyse the binding of the CD3 antibody to CD3 -expressing cells, where the CD3-expressing cells could be: CHOK1 cells overexpressing human CD3 or HEK 293cells (plasmids encoding the ORF of γ, δ, ε, and ζchains of human-derived CD3 and plasmids encoding the ORF of α and β chains of human TCR were co-transfected with host cells CHOK1 (ATCC, CCL-61) or HEK293 (ATCC, CRL-1573) to construct stable cell lines expressing the structure of the human TCR/CD3 complex); CHOK1 or HEK293 cells overexpressing cynomolgus monkey CD3; human pan-T cells (isolated with the human pan-T cell isolation kit (Miltenyi, #130-096-535) from PBMC); cynomolgus monkey pan-T cells. Specifically, the collected cells were washed twice with PBS containing 2% FBS (FACS buffer), and resuspended with FACS buffer, divided into 96 well plates with 1×10⁵ cells per well, centrifuged at 500 g for 5 minutes. The supernatant was discarded, and 100 μl of pre-gradient diluted CD3 antibody was added, then incubating for 1 hour at room temperature and washing twice with FACS buffer. The cells were resuspended with FACS buffer diluted with secondary antibody Alexa Fluor 488 AffiniPure Goat Anti-Human IgG, Fcγ fragment specific (Jackson ImmunoResearch, #109-545-098), then incubated at room temperature in the dark for 30 minutes. The cells were washed twice with FACS buffer, and resuspended with 200 μl FACS buffer. The fluorescent luminescence signal value were read by flow cytometry (BD FACS CANTOII or ACEA NovoCyte), and the resulted data were processed and analyzed by software FlowJo v10 (FlowJo, LLC). The software GraphPad Prism 8 was used for data processing and graphical analysis, and parameters such as binding curves and EC50 values can be obtained by four-parameter nonlinear fitting.

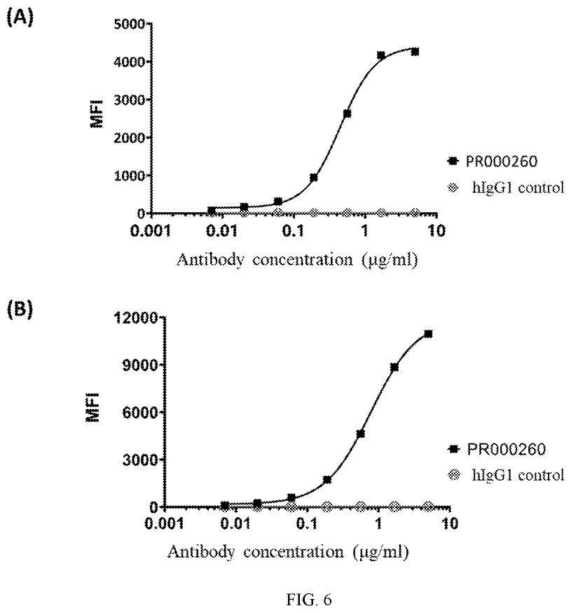

FIG. 6 shows the binding ability of the CD3 antibody obtained in the Example 2 to recombinant CHOK1 cells overexpressing human CD3 (FIG. 6(A)) and recombinant CHOK1 cells overexpressing cynomolgus monkey CD3 (FIG. 6(B)). The results indicate that the SP34 chimeric antibody PR000260 has a strong binding ability to both human CD3 and cynomolgus monkey CD3.

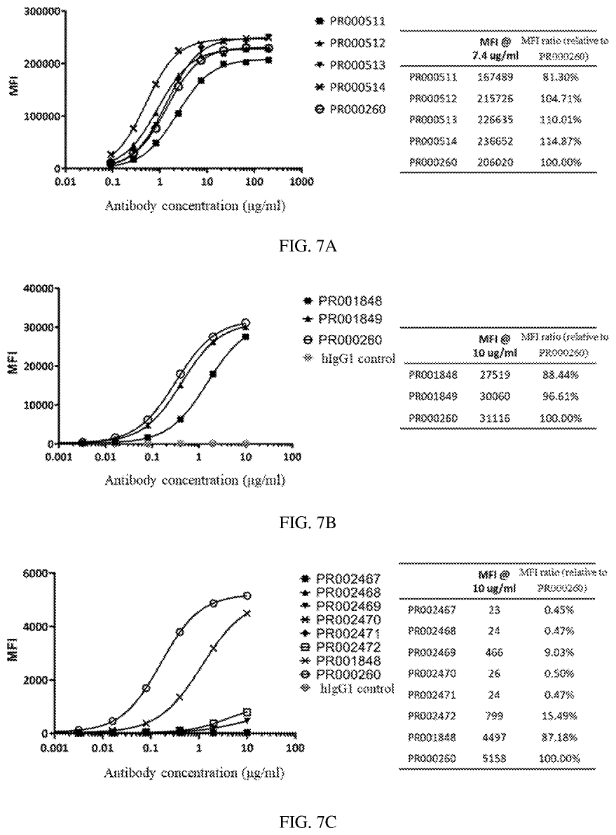

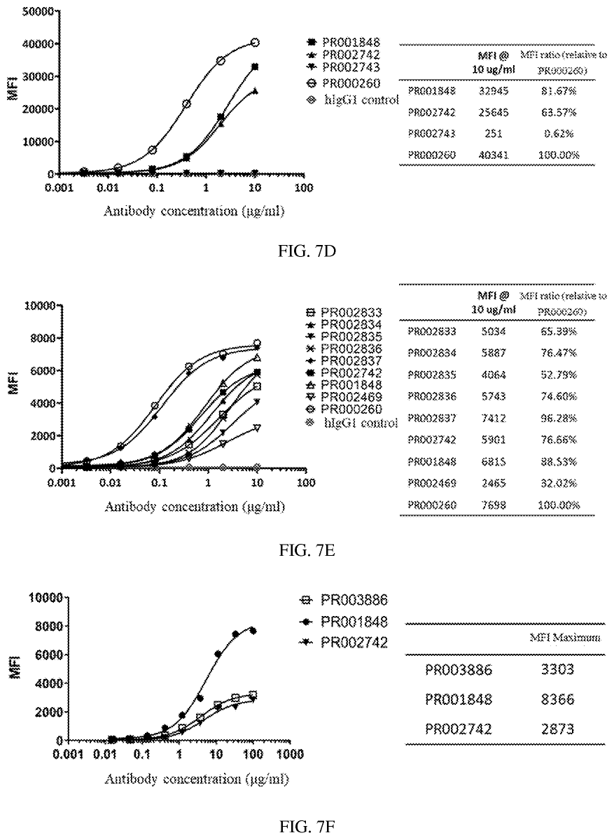

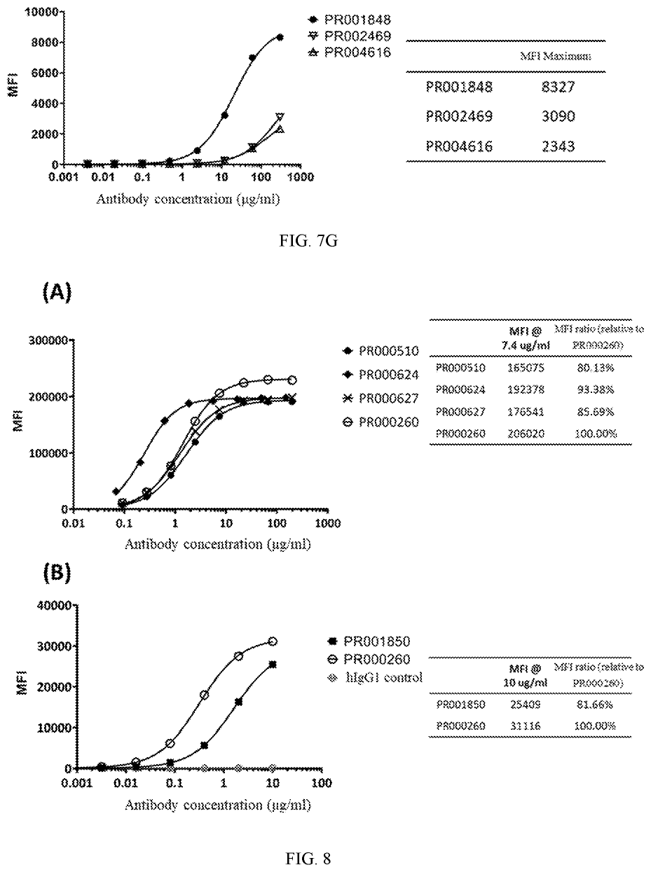

FIG. 7A to FIG. 7G show the binding ability of the CD3 antibodies obtained in the Example 4.2 (including PR000260 and its mutants) to human pan-T cells, respectively. The fluorescence intensity MFI of the CD3 antibody binding to human pan-T cells and the relative ratio relative to the initial antibody PR000260 were calculated when the antibody concentration was 7.4 or 10 μg/ml. Specifically, after SP34 IgG antibody sequence optimization, PR000512, PR000513, PR001849, and PR002837 have comparable binding ability as PR000260 (i.e., SP34 chimeric antibody); while PR000514 has slightly higher binding ability than PR000260; PR000511, PR001848 PR002469, PR002472, PR002742, PR002833, PR002834, PR002835, PR002836, PR003886, and PR004616 have lower binding ability to T cells; PR002467, PR002468, PR002470, PR002471, and PR002743, on the other hand, barely bind T cells (or the signal cannot be detected at the current antibody concentration). The above results indicate that the present disclosure has obtained several new antibodies by sequence optimization of the CD3 antibody, which have different binding abilities to human T cells and can be applied to different application scenarios.

FIG. 8(A) and (B) show the binding capacity of the anti-CD3 scFv-Fc single chain antibody obtained in the Example 4.3 to human pan-T cellsThe fluorescence intensity MFI of the CD3 antibody binding to human pan-T cells and the relative ratio relative to the initial antibody PR000260 were calculated when the antibody concentration was 7.4 or 10 μg/ml. Specifically, after optimization of the humanization of SP34 scFv antibody, PR000624 has a comparable or slightly higher binding capacity than PR000260; PR000510 and PR000627 have a comparable or slightly lower binding capacity than PR000260; and PR001850 has a significantly lower binding capacity to T cells than PR000260. The above results indicate that, the present disclosure also obtained several stable single-chain antibodies in the form of scFv by sequence optimization of the CD3 antibody, which are able to bind to human T cells and are suitable for application scenarios such as the construction of bispecific antibodies.

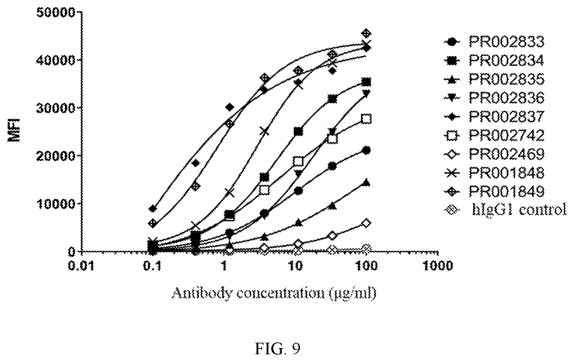

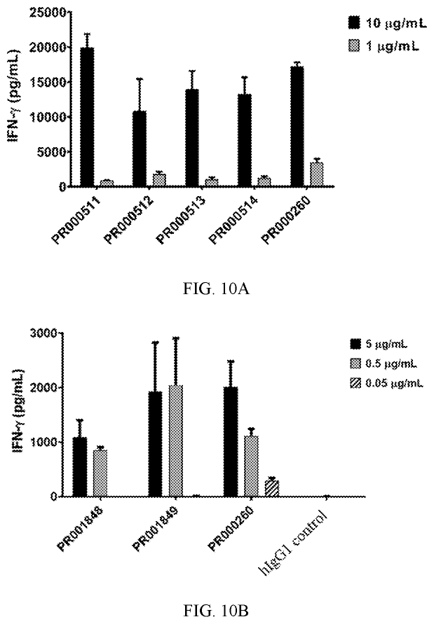

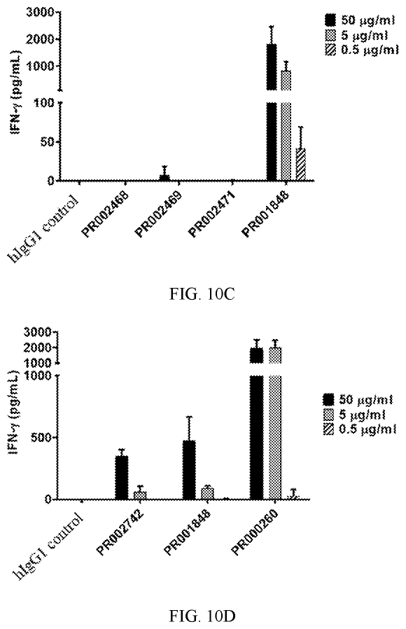

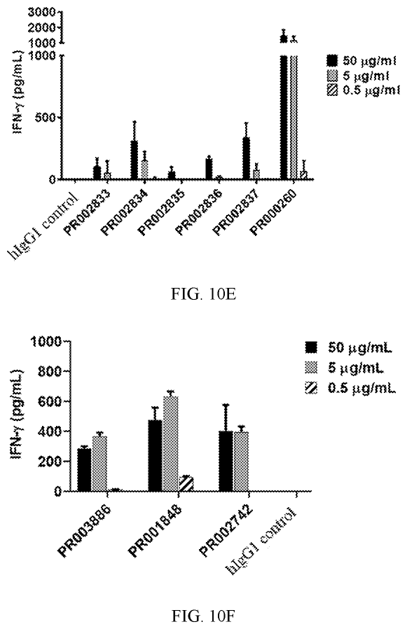

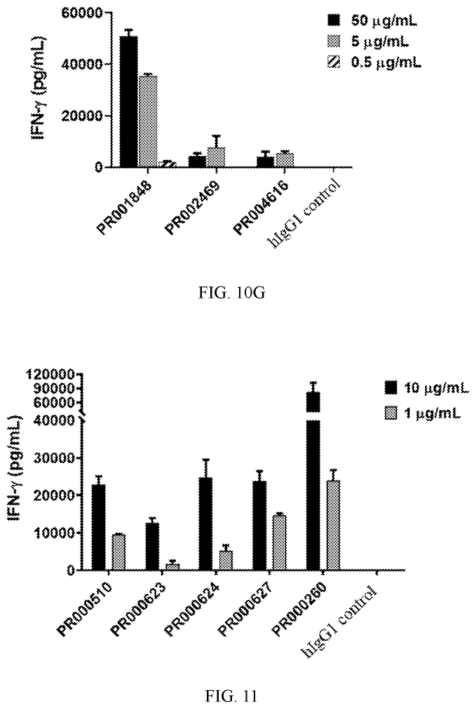

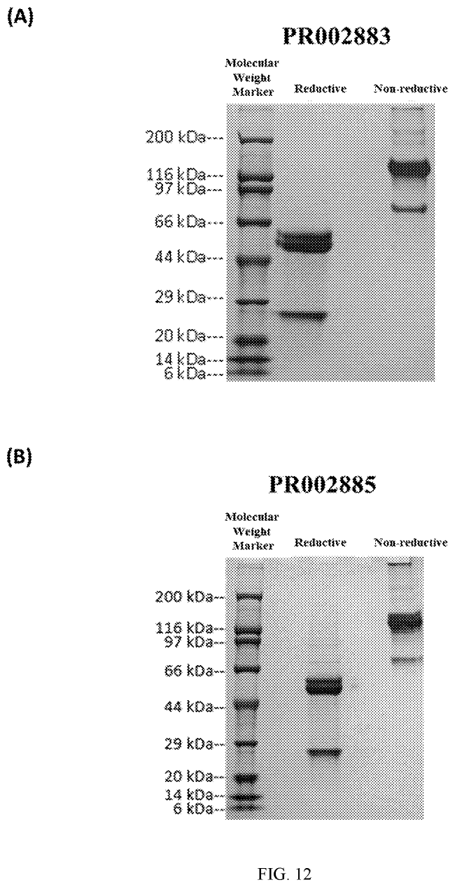

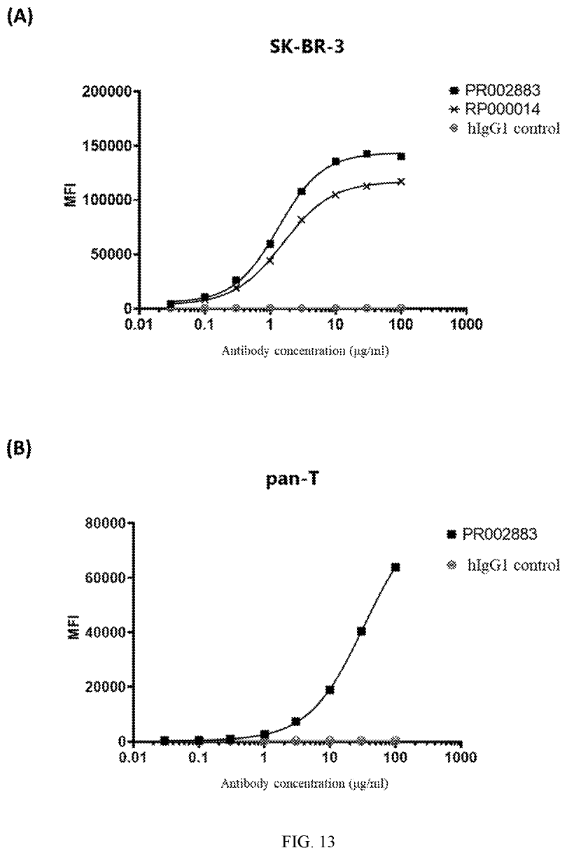

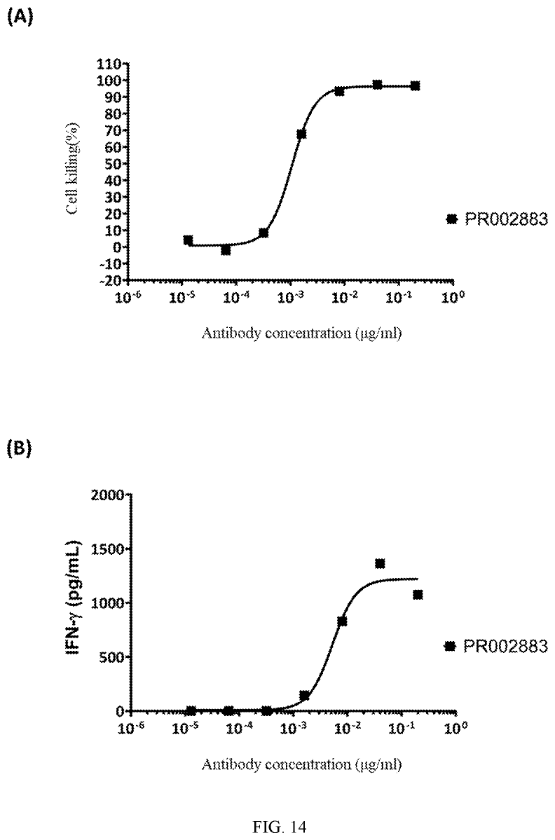

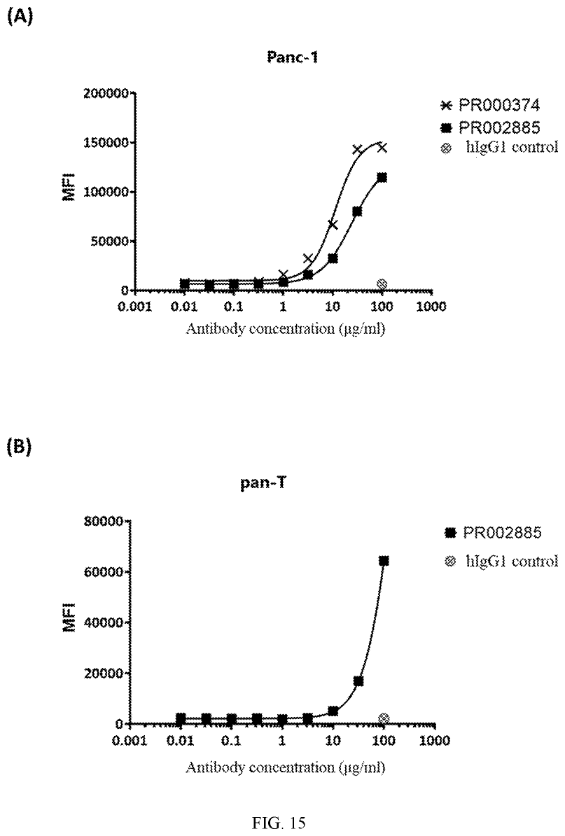

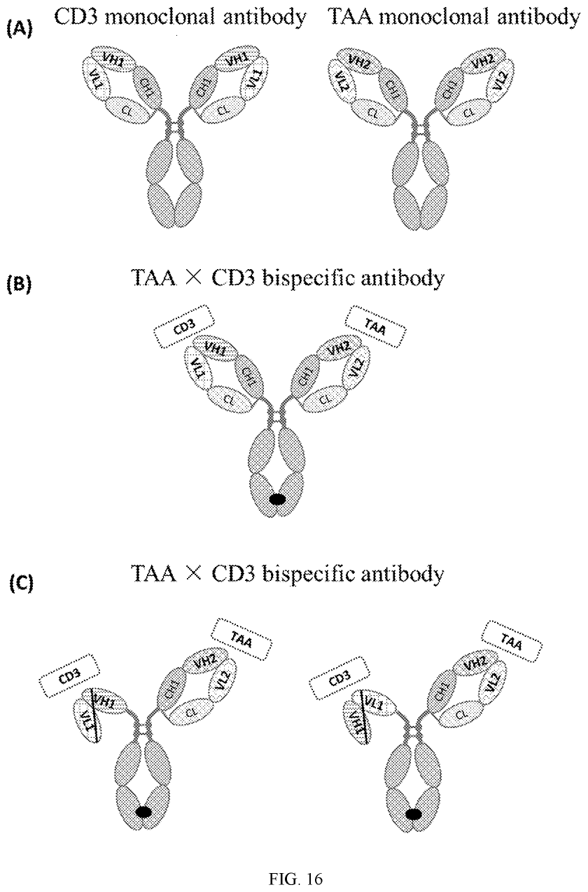

FIG. 9 shows the binding ability of some of the CD3 antibodies obtained in the Example 4.2 to cynomolgus monkey pan-T cells. It can be seen that different molecules have different binding abilities to cynomolgus monkey pan-T cells and are positively correlated with their binding abilities to human pan-T cells; that is, molecules that strongly bind to human pan-T cells also strongly bind to cynomolgus monkey pan-T cells, and vice versa.

### Example 6 Determination of Activation of CD3 Antibody on Human T Cells

The gradient dilutions of CD3 antibody (e.g., 50, 10, 5, 1, 0.5, 0.05 μg/ml) were coated in 96 well cell culture plates at three replicate wells per concentration and 50 μl per well, incubating overnight at 4° C. The cell density of human PBMC (MiaoTong Biology) or human pan-T cells (isolated with the human pan-T cell isolation kit (Miltenyi, #130-096-535) from PBMC) was adjusted to 7.5×10⁵/ml, and human CD28 antibody was added at a concentration of 1 μg/ml, thereafter adding 200 μl the resulted mixture per well to the cell culture plate and incubating in CO₂ incubator. After 72 hours of incubation, the supernatant was taken and the content of IFN-γ therein was determined using use the IFN-γ ELISA kit (Thermo, #88-7316-77). Used the software GraphPad Prism 8 for data processing and graphical analysis.

FIG. 10A to FIG. 10G show the ability of each CD3 antibody (including SP34 chimeric antibody) obtained in the Example 4.2 to activate human T cells, respectively. When the antibody concentration is 1 μg/mL, PR000511, PR000512, PR000513, and PR000514 produced significantly lower levels of IFN-γ after activating T cells than PR000260; when the antibody concentration is 10 μg/mL, PR000512, PR000513, and PR000514 activate slightly lower levels of IFN-γ than PR000260 (FIG. 10A). When the antibody concentration is 0.5 μg/mL and 5 μg/mL, the level of IFN-γ activated by PR001848 is significantly lower than that of PR000260 (FIG. 10(B)). In addition, the effect of activating T cells of antibodies PR002468, PR002469, PR002471, PR002742, PR002833, PR002834, PR002835, PR002836, PR002837, PR001848, PR003886 and PR004616 was also detected (FIG. 10C to FIG. 10G), in concentration of 0.5 μg/mL, 5 μg/mL and 50 μg/mL, the results indicated that the level of IFN-γ produced by T cells activated by these antibodies are much lower than those activated by PR000260, wherein, no release of IFN-γ was detected by T cells activated by PR002468 and PR002471, and only weak level of IFN-γ were detected at 50 μg/mL of PR002469 and PR002835; the level of IFN-γ produced by T cells activated by PR002742 and PR003886 were comparable and slightly weaker than PR001848; the level of IFN-γ produced by T cells activated by PR002469 and PR004616 were comparable and significantly weaker than PR001848. The above results indicate that the present disclosure has obtained several new antibodies by sequence optimization of the CD3 antibody, which have different activation abilities on human T cells, and can control different levels of cytokine release and can be applied to different application scenarios.

FIG. 11 shows the ability of the anti-CD3 scFv-Fc antibody obtained in the Example 4.3 to activate human T cells. PR000510, PR000623, PR000624, and PR000627 at concentrations of 1 μg/mL and 10 μg/mL all exhibited lower levels of IFN-γ than PR000260 but higher than the isotype control antibody, indicating that these four molecules limited the release of cytokines by regulating the activation levels of T cells. The above results indicate that the present disclosure also obtained several stable single-chain antibodies in the form of scFv by sequence optimization of the CD3 antibody, which have weaker activation on human T cells showing lower levels of cytokine release, and are suitable for application scenarios such as the construction of bispecific antibodies.

### Example 7 Bispecific Antibody Targeting B7H4 Containing Anti-CD3 scFv Antibody

B7H4, a member of the B7 family of transmembrane proteins, is highly expressed in a variety of solid tumor tissues such as breast, ovarian and endometrial cancers, while it is not expressed or very faintly expressed in normal tissues, making B7H4 a very specific tumor-associated target antigen. A bispecific antibody molecule targeting both B7H4 and CD3 were constructed, which can selectively activate T cells near tumor cells by targeting and binding to B7H4 on the surface of tumor cells, thus providing specific killing of tumor cells.

### 7.1 Preparation of B7H4 Antibody

The sequence of the variable region of the B7H4 antibody can be derived from WO2016040724, and the recombinant IgG antibody PR000014 target B7H4 was constructed according to the methods of the Example 1.1. The following Table 10 lists the sequence information of the B7H4 antibody PR000014.

TABLE 10
Sequence information of light chain and
heavy chain of B7H4 antibody PR000014

| Antibody | | Heavy chain | Light chain |
|---|---|---|---|
| number | Target | SEQ ID NO: | SEQ ID NO: |
| PR000014 | B7H4 | 82 | 83 |

7.2 Preparation of Bispecific Antibody Targeting B7H4 Containing Anti-CD3 scFv Antibody

Using the sequence of the B7H4 antibody PR000014 obtained in the Example 7.1 and the sequence of the CD3 single chain antibody PR000627 obtained in the Example 4.3 to construct the bispecific antibody molecule PR002883 targeting B7H4×CD3, which contains three polypeptide chains: a heavy chain containing the CD3 single chain antibody scFv (SEQ ID NO: 88), a heavy chain containing the VH of B7H4 antibody (SEQ ID NO: 86), and a light chain containing the VL of B7H4 antibody (SEQ ID NO: 83). The structure is shown in FIG. 16(C). Since the molecule has a special asymmetric structure, different amino acid mutations were introduced in the constant regions of the two heavy chains in order to reduce the generation of homologous heavy chain dimers. At the same time, the “LALAPG” triple mutation (L234A/L235A/P329G) was introduced in the constant region of the heavy chain to prevent cross-linking and reduce effector function caused by Fcγ receptor binding.

Recombinant protein of the bispecific antibody PR002883 was prepared by using the method described in the Example 1.1 in combination with plasmids in ratio (e.g., 1:1:1 or other ratios), and the follow-up one-step affinity purification. The sequence of bispecific antibody PR002883 is listed in Table 11; the expression of the bispecific antibody is listed in Table 12.

TABLE 11
Chains of the bispecific antibody and
the corresponding sequence information

| | | | Heavy | Heavy | Light |
|---|---|---|---|---|---|
| | Anti- | Anti- | Chain 1 | Chain 2 | chain |
| Bispecific | B7H4 | CD3 | SEQ ID | SEQ ID | SEQ ID |
| antibodies | antibody | scFv | NO: | NO: | NO: |
| PR002883 | PR000014 | PR000627 | 88 | 86 | 83 |

TABLE 12
Expression of the bispecific antibody

| Bispecific | Yield in | SDS-PAGE |
|---|---|---|
| antibody | HEK293 (mg/L) | purity (%) |
| PR002883 | 94.0 | 70 |

FIG. 12(A) shows the results of the bispecific antibody PR002883 after one-step purification by SDS-PAGE analysis. It shows that its main by-products are incompletely assembled molecules with few high polymer components, which can be reduced by optimizing the purification step or by optimizing the plasmid transfection ratio.

### 7.3 Binding to Tumor Cells Expressing B7H4

This Example investigates the ability of the bispecific antibody binding tumor cell SK-BR-3 (ATCC, HTB-30) expressing human B7H4. Specifically, collected cell SK-BR-3 suspension, adjusted the cell density to 1×10⁶/ml, and inoculated them at 100 μl/well in a 96 well V-bottom plate (Corning, #3894); subsequently, the antibody to be tested with a concentration 2-fold of final concentration obtained by 3-fold gradient dilution was added at volume of 100 μl/well. Cells were incubated at 4° C. in the dark for 2 hours. Afterwards, the cells were rinsed twice with 100 μl/well of pre-chilled PBS, centrifuged at 500 g, 4° C. for 5 minutes. The supernatant was discarded. Then added 100 μl/well of secondary antibody Alexa Fluor 488 AffiniPure Goat Anti-Human IgG, Fcγ fragment specific (Jackson ImmunoResearch, #109-545-098), and incubated the cells from light at 4° C. in the dark for 1 hour. Then the cells were rinsed twice with 100 μl/well of pre-chilled PBS, centrifuged at 500 g, for 5 minutes. The supernatant was discarded. Finally, the cells were resuspended with 200 μL/well of pre-chilled PBS. The fluorescent luminescence signal value were read by flow cytometry (BD FACS CANTOII or ACEA NovoCyte), and the resulted data were processed and analyzed by software FlowJo v10 (FlowJo, LLC). The software GraphPad Prism 8 was used for data processing and graphical analysis, and parameters such as binding curves and EC50 values can be obtained by four-parameter nonlinear fitting.

FIG. 13(A) shows the binding ability of the monoclonal antibody obtained in the Example 7.1 and the bispecific antibody obtained in the Example 7.2 to cell SK-BR-3. It can be seen that the bispecific antibody PR002883 has a comparable or even better binding capacity compared to the monoclonal antibody PR000014.

### 7.4 Binding to Human T Cells

The ability of the bispecific antibody PR002883 binding to human pan-T cells was detected by the method described in the Example 5. As shown in FIG. 13(B), PR002883 is able to bind to human pan-T cells.

### 7.5 Killing and Cytokine Releasing Effect of the Bispecific Antibody on B7H4 High-Expressing Cell Line SK-BR-3 In Vitro

To investigate the ability of killing target cell mediated by B7H4×CD3 bispecific antibody in vitro, human PBMC was used as effector cells, and B7H4 high-express cell line SK-BR-3 (ATCC, HTB-30) was used as target cells for killing assays in vitro and cytokine release detection. Specifically, 50 μL of RPMI1640/10% FBS medium was added to each well of E-plate (ACEA Biosciences Inc., #05232368001), thereafter balancing in incubator containing 5% CO₂ at 37° C. for 30 minutes, and then the E-plate was placed in the instrument xCELLigence RTCA (ACEA Biosciences) to test for normality. The density of SK-BR-3 was adjusted to 0.4×10⁶ cells/mL with RPMI1640/10% FBS medium, then inoculated into E-plate at 50 μL cells/well, and then the E-plate was placed on xCELLigence RTCA overnight to detect the cell index. Thedensity of PBMC was adjusted to 4×10⁶ cells/mL with RPMI1640/10% FBS medium, and inoculated into E-plate at 50 μL cells/well, then the antibody to be tested with a concentration 4-flod of final concentration obtained by 5-fold gradient dilution was added at 50 μL/well, wherein the highest final concentration of the antibody was 0.2 nM, and there were 7 concentrations for each antibody, the final ratio of effector cells to target cells was 10:1, with two replicates set up. Meanwhile, blank control was set up in the plate: SKBR3+PBMC+RPMI1640/10% FBS medium; the E-plate was incubated in 37° C., incubator containing 5% CO₂ for 24 hours. After incubation, the E-plate was placed on the xCELLigence RTCA instrument to detect the cell index.

The specific killing effect of the antibody was calculated by applying the assayed cell index to the following formula:

$$Cell Killing \% = ( 1 - Test Sample / Blank Control ) * 100 \% .$$

The supernatant of cell culture was collected for detecting the release of cytokine IFN-γ. Refer to the operation instructions of IFN-γ kit (IFN gamma Human Uncoated ELISA Kit, Thermo, #88-7316-77) for ELISA detection.

As shown in FIG. 14(A) and (B), the bispecific antibody PR002883 can activate T cells to release cytokines such as IFN-γ and kill tumor cell SK-BR-3 effectively. Almost 100% of tumor cells were killed when the concentration of the bispecific antibody was at 0.01 μg/ml (FIG. 14(A)).

### Example 8 Bispecific Antibody Targeting ROR1 Containing Anti-CD3 scFv Antibody

ROR1 is an inactive tyrosine protein kinase transmembrane protein that is overexpressed in many tumors but virtually not expressed in normal tissues. ROR1 contributes to cell proliferation and migration in chronic lymphocytic leukemia by transducing the Wnt signaling pathway after interacting with Wnt5a as a receptor, and contributes to epithelial-mesenchymal-transformation (EMT) in solid tumors. The tumor-specific expression of ROR1 makes it a suitable tumor-associated antigen target for the development of therapeutic agents. The construction of a bispecific antibody molecule targeting both ROR1 and CD3 can selectively activate T cells near tumor cells by targeting and binding to ROR1 on the surface of tumor cells, thus providing specific killing of tumor cells.

### 8.1 Preparation of ROR1 Antibody

The sequence of the variable region of the ROR1 antibody can be derived from WO2016094873, and construct the recombinant IgG antibody PR000374 targeting ROR1 according to the methods of the Example 1.1. The following Table 13 lists the sequence information of the ROR1 antibody PR000374.

TABLE 13
Sequence list of the ROR1 antibody PR000374.

| Antibody | | Heavy chain | Light chain |
|---|---|---|---|
| number | Target | SEQ ID NO: | SEQ ID NO: |
| PR000374 | ROR1 | 84 | 85 |

8.2 Preparation of Bispecific Antibody Targeting ROR1 Containing Anti-CD3 scFv Antibody

Construction of the bispecific antibody molecule PR002885 targeting ROR1×CD3 was performed by using the sequence of the ROR1 antibody PR000374 obtained in the Example 8.1 and the sequence of the CD3 single chain antibody PR000627 obtained in the Example 4.3, which contains three polypeptide chains: a heavy chain containing the CD3 single chain antibody scFv (SEQ ID NO: 88), a heavy chain containing the ROR1 antibody VH (SEQ ID NO: 87), and a light chain containing the ROR1 antibody VL (SEQ ID NO: 85). The structure is shown in FIG. 16(C). Since the molecule has a special asymmetric structure, different amino acid mutations were introduced into the constant regions of the two heavy chains in order to reduce the generation of homologous heavy chain dimers. At the same time, the “LALAPG” triple mutation (L234A/L235A/P329G) was introduced into the constant region of the heavy chain to prevent cross-linking and reduce effector function caused by Fcγ receptor binding.

Recombinant protein of the bispecific antibody PR002885 was prepared by using the method described in the Example 1.1 in combination with plasmids ratio (e.g., 1:1:1 or other ratios), and the follow-up one-step affinity purification. The sequence of bispecific antibody PR002885 is listed in Table 14; the expression of the bispecific antibody is listed in Table 15.

TABLE 14
Chains of the bispecific antibody and
the corresponding sequence number

| | | | Heavy | Heavy | Light |
|---|---|---|---|---|---|
| | Anti- | Anti- | chain1 | chain2 | chain |
| Bispecific | ROR1 | CD3 | SEQ ID | SEQ ID | SEQ ID |
| antibody | antibody | scFv | NO: | NO: | NO: |
| PR002885 | PR000374 | PR000627 | 88 | 87 | 85 |

TABLE 15
Expression of the bi specific antibody

| Bispecific | Yield in | SDS-PAGE |
|---|---|---|
| antibody | HEK293 (mg/L) | purity (%) |
| PR002885 | 90.0 | 70 |

FIG. 12(B) shows the results of the bispecific antibody PR002885 after one-step purification by SDS-PAGE analysis. It shows that its main by-products are incompletely assembled molecules with few high polymer components, which can be reduced by optimizing the purification step or by optimizing the plasmid transfection ratio.

### 8.3 Binding to Tumor Cells Expressing ROR1

This Example investigates the ability of the bispecific antibody binding tumor cell Panc-1 (ATCC, CRL-1469) expressing human ROR1. Specifically, collected cell Panc-1 suspension, adjusted the cell density to 1×10⁶/ml, and inoculated them at 100 μl/well in a 96 well V-bottom plate (Corning, #3894); subsequently, the antibody to be tested with a concentration 2-flod of final concentration obtained by 3-fold gradient dilution was added at volume of 100 μl/well. Cells were incubated at 4° C. in the dark for 2 hours. Afterwards, the cells were rinsed twice with 100 μl/well of pre-chilled PBS, centrifuged at 500 g, 4° C. for 5 minutes. The supernatant was discarded. Then added 100 μl/well of fluorescent secondary antibody Alexa Fluor 488 AffiniPure Goat Anti-Human IgG, Fcγ fragment specific (Jackson ImmunoResearch, #109-545-098), and incubated the cells from light at 4° C. in the dark for 1 hour. Then the cells were rinsed twice with 100 μl/well of pre-chilled PBS, centrifuged at 500 g, for 5 minutes. The supernatant was discarded. Finally, the cells were resuspended with 200 μL/well of pre-chilled PBS. The fluorescent luminescence signal value were read by flow cytometry (BD FACS CANTOII or ACEA NovoCyte), and the resulted data were processed and analyzed by software FlowJo v10 (FlowJo, LLC). The software GraphPad Prism 8 was used for data processing and graphical analysis, and parameters such as binding curves and EC50 values can be obtained by four-parameter nonlinear fitting.

FIG. 15(A) shows the binding ability of the monoclonal antibody obtained in the Example 8.1 and the bispecific antibody obtained in the Example 8.2 to cell Panc-1. It can be seen that both the bispecific antibody PR002885 and the monoclonal antibody PR000374 can bind Panc-1.

### 8.4 Binding to Human T Cells

The ability of the bispecific antibody PR002885 binding to human pan-T cells was detected by the method described in the Example 5. As shown in FIG. 15(B), PR002885 is able to bind to human pan-T cells.

What is claimed is:

1. A CD3-targeting antibody, comprising a light chain variable region (VL) and a heavy chain variable region (VH); wherein the amino acid sequence of the VL is set forth in SEQ ID NO: 56 or a mutant thereof, wherein the mutant comprises one or more mutations at positions 2,36,46,49,66, 69, 71 and 87 compared to SEQ ID NO: 56, according to Chothia numbering scheme.

2. The CD3-targeting antibody of claim 1, wherein the VH has mutations selected from the following groups:
(a) A2I;
(b) V36F or V36Y;
(c) G46T or G46L;
(d) G49Y;
(e) L66G;
(f) D69G or D69T;
(g) A71F; and
(h) F87Y.

3. The CD3-targeting antibody of claim 1, wherein the amino acid sequence of the VL is set forth in any one of SEQ ID NOs: 60,57 and 59; preferably,
wherein the amino acid sequence of the VH is set forth in any one of SEQ ID NOs: 43,44 and 51.

4. The CD3-targeting antibody of claim 3, wherein,
the amino acid sequence of the VH is set forth in SEQ ID NO: 44, and the amino acid sequence of the VL is set forth in SEQ ID NO: 60; or,
the amino acid sequence of the VH is set forth in SEQ ID NO: 51, and the amino acid sequence of the VL is set forth in SEQ ID NO: 60; or,
the amino acid sequence of the VH is set forth in SEQ ID NO: 43, and the amino acid sequence of the VL is set forth in SEQ ID NO: 60; or,
the amino acid sequence of the VH is set forth in SEQ ID NO: 43, and the amino acid sequence of the VL is set forth in SEQ ID NO: 57; or,
the amino acid sequence of the VH is set forth in SEQ ID NO: 44, and the amino acid sequence of the VL is set forth in SEQ ID NO: 57; or,
the amino acid sequence of the VH is set forth in SEQ ID NO: 51, and the amino acid sequence of the VL is set forth in SEQ ID NO: 57; or,
the amino acid sequence of the VH is set forth in SEQ ID NO: 43, and the amino acid sequence of the VL is set forth in SEQ ID NO: 59; or,
the amino acid sequence of the VH is set forth in SEQ ID NO: 44, and the amino acid sequence of the VL is set forth in SEQ ID NO: 59.

5. The CD3-targeting antibody of claim 1, wherein the antibody comprises a single chain antibody (scFv) of VL-linker-VH or VH-linker-VL.

6. The CD3-targeting antibody of claim 5, wherein the linker is (G4S) n or a variant thereof, wherein n is a non-zero natural number, preferably 1 to 20; more preferably the amino acid sequence of the linker is set forth in SEQ ID NO: 65, SEQ ID NO: 66, or SEQ ID NO: 67.

7. The CD3-targeting antibody of claim 6, wherein the amino acid sequence of the scFv is set forth in SEQ ID NO: 74, SEQ ID NO: 75, SEQ ID NO: 76 or SEQ ID NO: 77.

8. The CD3-targeting antibody of claim 7, wherein the antibody further comprises Fc, wherein the Fc is linked to the scFv by a hinge.

9. The CD3-targeting antibody of claim 1, wherein the antibody further comprises a constant region, preferably a human constant region.

10. The CD3-targeting antibody of claim 9, wherein the constant region is a human constant region comprising a human light chain constant region and a human heavy chain constant region, and the human light chain constant region is preferably a human k light chain constant region as shown in SEQ ID NO: 61 or a human 2 light chain constant region as shown in SEQ ID NO: 62.

11. The CD3-targeting antibody of claim 10, wherein the human heavy chain constant region is from hIgG1, hIgG2, hIgG3, hIgG4, or a variant thereof, preferably a heavy chain constant region as shown in SEQ ID NO: 63 or SEQ ID NO: 64.

12. A bispecific antibody, comprising a first protein functional region and a second protein functional region, wherein the first protein functional region comprises the CD3-targeting antibody of claim 1.

13. The bispecific antibody of claim 12, wherein the bispecific antibody comprises the following three chains: (1) VL1-linker-VH1-Hinge-CH2-CH3 (knob) or VH1-linker-VL1-Hinge-CH2-CH3 (knob) of the first protein functional region, (2) VH2-CH1-Hinge-CH2-CH3 (hole) of the second protein functional region, and (3) VL2-CL of the second protein functional region; the second protein functional region is a non-CD3-targeting antibody, preferably a B7H4-targeting antibody or a ROR1-targeting antibody, and the linker is preferably (G4S) n, wherein n is a non-zero natural number, preferably 1 to 20, and more preferably the amino acid sequence of the linker is set forth in SEQ ID NO: 65, SEQ ID NO: 66, or SEQ ID NO: 67.

14. The bispecific antibody of claim 13, wherein the bispecific antibody comprises VL1-linker-VH1-Hinge-CH2-CH3 (knob) as shown in SEQ ID NO: 88, VH2-CH1-Hinge-CH2-CH3 (hole) as shown in SEQ ID NO: 86, and VL2-CL as shown in SEQ ID NO: 83, or, VL1-linker-VH1-Hinge-CH2-CH3 (knob) as shown in SEQ ID NO: 88, VH2-CH1-Hinge-CH2-CH3 (hole) as shown in SEQ ID NO: 87, and VL2-CL as shown in SEQ ID NO: 85.

15. An isolated nucleic acid, encoding the CD3-targeting antibody of claim 1 or a bispecific antibody wherein the bispecific antibody comprising a first protein functional region and a second protein functional region, wherein the first protein functional region comprises the CD3 -targeting antibody.

16. An expression vector, comprising the isolated nucleic acid of claim 15; preferably, the expression vector is selected from a retroviral vector, a lentiviral vector, an adenovirus vector, and an adeno-associated virus vector.

17. A genetically modified cell, transfected with the expression vector of claim 16;
preferably, the genetically modified cell is a eukaryotic cell.

18. A pharmaceutical composition, comprising the CD3-targeting antibody of claim 1, a bispecific antibody, and a pharmaceutically acceptable carrier;
wherein the bispecific antibody comprises a first protein functional region and a second protein functional region, wherein the first protein functional region comprises the CD3-targeting antibody;
preferably, the pharmaceutical composition further comprises an immune checkpoint antibody.

19. A method for treating a patient in need of a medicament for tumor, comprising administering to the patient a medicament comprising an effective amount of the CD3-targeting antibody of claim 1, a bispecific antibody or a pharmaceutical composition;
wherein the bispecific antibody comprising a first protein functional region and a second protein functional region, wherein the first protein functional region comprises the CD3-targeting antibody;
wherein the pharmaceutical composition comprising the CD3-targeting antibody, a bispecific antibody and a pharmaceutically acceptable carrier;
preferably, the pharmaceutical composition further comprises an immune checkpoint antibody.


SEQUENCE LISTING (92 sequences)

SEQ ID NO: 1 - protein, 7 aa, synthetic construct
  REGION 1..7: note = SP34_VH|HCDR1|Chothia; VH3230|HCDR1|Chothia; VH3231|HCDR1|Chothia; VH3
GFTFNTY

SEQ ID NO: 2 - protein, 7 aa, synthetic construct
  REGION 1..7: note = VH3232|HCDR1|Chothia; VH3233|HCDR1|Chothia; VH3234|HCDR1|Chothia; VH32
GFTFSTY

SEQ ID NO: 3 - protein, 8 aa, synthetic construct
  REGION 1..8: note = SP34_VH|HCDR2|Chothia; VH3230|HCDR2|Chothia; VH3231|HCDR2|Chothia; VH3
RSKYNNYA

SEQ ID NO: 4 - protein, 14 aa, synthetic construct
  REGION 1..14: note = SP34_VH|HCDR3|Chothia; VH3230|HCDR3|Chothia; VH3231|HCDR3|Chothia; VH3
HGNFGNSYVSWFAY

SEQ ID NO: 5 - protein, 25 aa, synthetic construct
  REGION 1..25: note = SP34_VH|HFWR1|Chothia
EVQLVESGGGLVQPKGSLKLSCAAS

SEQ ID NO: 6 - protein, 25 aa, synthetic construct
  REGION 1..25: note = VH3230|HFWR1|Chothia; VH3231|HFWR1|Chothia; VH3232|HFWR1|Chothia; VH32
EVQLLESGGGLVQPGGSLRLSCAAS

SEQ ID NO: 7 - protein, 25 aa, synthetic construct
  REGION 1..25: note = VH3730|HFWR1|Chothia; VH3731|HFWR1|Chothia; VH3732|HFWR1|Chothia; VH37
EVQLVESGGGLVQPGGSLKLSCAAS

SEQ ID NO: 8 - protein, 19 aa, synthetic construct
  REGION 1..19: note = SP34_VH|HFWR2|Chothia
AMNWVRQAPGKGLEWVARI

SEQ ID NO: 9 - protein, 19 aa, synthetic construct
  REGION 1..19: note = VH3230|HFWR2|Chothia; VH3231|HFWR2|Chothia; VH3232|HFWR2|Chothia; VH32
AMNWVRQAPGKGLEWVSRI

SEQ ID NO: 10 - protein, 19 aa, synthetic construct
  REGION 1..19: note = VH3730|HFWR2|Chothia; VH3731|HFWR2|Chothia; VH3732|HFWR2|Chothia; VH37
AMNWVRQASGKGLEWVGRI

SEQ ID NO: 11 - protein, 41 aa, synthetic construct
  REGION 1..41: note = SP34_VH|HFWR3|Chothia
TYYADSVKDRFTISRDDSQSILYLQMNNLKTEDTAMYYCVR

SEQ ID NO: 12 - protein, 41 aa, synthetic construct
  REGION 1..41: note = VH3230|HFWR3|Chothia; VH3233|HFWR3|Chothia
TYYADSVKDRFTISRDNSKNTLYLQMNSLRAEDTAVYYCAK

SEQ ID NO: 13 - protein, 41 aa, synthetic construct
  REGION 1..41: note = VH3231|HFWR3|Chothia; VH3232|HFWR3|Chothia
TYYADSVKDRFTISRDDSKSTLYLQMNSLRAEDTAVYYCVR

SEQ ID NO: 14 - protein, 41 aa, synthetic construct
  REGION 1..41: note = VH3234|HFWR3|Chothia
TYYADSVKDRFTISRDNSKNTLYLQMNSLRAEDTAVYYCVR

SEQ ID NO: 15 - protein, 41 aa, synthetic construct
  REGION 1..41: note = VH3235|HFWR3|Chothia
TYYADSVKDRFTISRDDSKSTLYLQMNSLRAEDTAVYYCAK

SEQ ID NO: 16 - protein, 41 aa, synthetic construct
  REGION 1..41: note = VH3236|HFWR3|Chothia
TYYADSVKDRFTISRDNSKSTLYLQMNSLRAEDTAVYYCAR

SEQ ID NO: 17 - protein, 41 aa, synthetic construct
  REGION 1..41: note = VH3730|HFWR3|Chothia; VH3735|HFWR3|Chothia
TYYADSVKDRFTISRDDSKNTAYLQMNSLKTEDTAVYYCTR

SEQ ID NO: 18 - protein, 41 aa, synthetic construct
  REGION 1..41: note = VH3731|HFWR3|Chothia; VH3732|HFWR3|Chothia
TYYADSVKDRFTISRDDSKSTLYLQMNSLKTEDTAVYYCVR

SEQ ID NO: 19 - protein, 41 aa, synthetic construct
  REGION 1..41: note = VH3733|HFWR3|Chothia
TYYADSVKDRFTISRDDSKSTLYLQMNSLKTEDTAVYYCTR

SEQ ID NO: 20 - protein, 41 aa, synthetic construct
  REGION 1..41: note = VH3734|HFWR3|Chothia
TYYADSVKDRFTISRDDSKNTAYLQMNSLKTEDTAVYYCVR

SEQ ID NO: 21 - protein, 11 aa, synthetic construct
  REGION 1..11: note = SP34_VH|HFWR4|Chothia
WGQGTLVTVSA

SEQ ID NO: 22 - protein, 11 aa, synthetic construct
  REGION 1..11: note = VH3230|HFWR4|Chothia; VH3231|HFWR4|Chothia; VH3232|HFWR4|Chothia; VH32
WGQGTLVTVSS

SEQ ID NO: 23 - protein, 14 aa, synthetic construct
  REGION 1..14: note = SP34_VL|LCDR1|Chothia; VK1392|LCDR1|Chothia; VK1393|LCDR1|Chothia; VL7
RSSTGAVTTSNYAN

SEQ ID NO: 24 - protein, 7 aa, synthetic construct
  REGION 1..7: note = SP34_VL|LCDR2|Chothia; VK1392|LCDR2|Chothia; VK1393|LCDR2|Chothia; VL7
GTNKRAP

SEQ ID NO: 25 - protein, 9 aa, synthetic construct
  REGION 1..9: note = SP34_VL|LCDR3|Chothia; VK1392|LCDR3|Chothia; VK1393|LCDR3|Chothia; VL7
ALWYSNLWV

SEQ ID NO: 26 - protein, 22 aa, synthetic construct
  REGION 1..22: note = SP34_VL|LFWR1|Chothia
QAVVTQESALTTSPGETVTLTC

SEQ ID NO: 27 - protein, 22 aa, synthetic construct
  REGION 1..22: note = VL7460|LFWR1|Chothia; VL7461|LFWR1|Chothia
QAVVTQEPSLTVSPGGTVTLTC

SEQ ID NO: 28 - protein, 23 aa, synthetic construct
  REGION 1..23: note = VK1392|LFWR1|Chothia
DIQMTQSPSSLSASVGDRVTITC

SEQ ID NO: 29 - protein, 23 aa, synthetic construct
  REGION 1..23: note = VK1393|LFWR1|Chothia
DAQMTQSPSSLSASVGDRVTITC

SEQ ID NO: 30 - protein, 15 aa, synthetic construct
  REGION 1..15: note = SP34_VL|LFWR2|Chothia
WVQEKPDHLFTGLIG

SEQ ID NO: 31 - protein, 15 aa, synthetic construct
  REGION 1..15: note = VK1392|LFWR2|Chothia
WYQQKPGKAPKLLIY

SEQ ID NO: 32 - protein, 15 aa, synthetic construct
  REGION 1..15: note = VK1393|LFWR2|Chothia
WVQQKPGKAPKGLIG

SEQ ID NO: 33 - protein, 15 aa, synthetic construct
  REGION 1..15: note = VL7460|LFWR2|Chothia
WFQQKPGQAPRTLIY

SEQ ID NO: 34 - protein, 15 aa, synthetic construct
  REGION 1..15: note = VL7461|LFWR2|Chothia
WVQQKPGQAPRGLIG

SEQ ID NO: 35 - protein, 32 aa, synthetic construct
  REGION 1..32: note = SP34_VL|LFWR3|Chothia
GVPARFSGSLIGDKAALTITGAQTEDEAIYFC

SEQ ID NO: 36 - protein, 32 aa, synthetic construct
  REGION 1..32: note = VK1392|LFWR3|Chothia
GVPSRFSGSGSGTDFTLTISSLQPEDFATYYC

SEQ ID NO: 37 - protein, 32 aa, synthetic construct
  REGION 1..32: note = VK1393|LFWR3|Chothia
GVPSRFSGSLSGDDATLTISSLQPEDFATYFC

SEQ ID NO: 38 - protein, 32 aa, synthetic construct
  REGION 1..32: note = VL7460|LFWR3|Chothia
WTPARFSGSLLGGKAALTLLGAQPEDEAEYYC

SEQ ID NO: 39 - protein, 32 aa, synthetic construct
  REGION 1..32: note = VL7461|LFWR3|Chothia
WTPARFSGSLLGDKAALTLLGAQPEDEAEYFC

SEQ ID NO: 40 - protein, 10 aa, synthetic construct
  REGION 1..10: note = SP34_VL|LFWR4|Chothia; VL7460|LFWR4|Chothia; VL7461|LFWR4|Chothia
FGGGTKLTVL

SEQ ID NO: 41 - protein, 10 aa, synthetic construct
  REGION 1..10: note = VK1392|LFWR4|Chothia; VK1393|LFWR4|Chothia
FGGGTKVEIK

SEQ ID NO: 42 - protein, 125 aa, synthetic construct
  REGION 1..125: note = SP34_VH|VH|Chothia
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 43 - protein, 125 aa, synthetic construct
  REGION 1..125: note = VH3230|VH|Chothia
EVQLLESGGGLVQPGGSLRLSCAASGFTFNTYAMNWVRQAPGKGLEWVSRIRSKYNNYATYYADSVKDRFTISRDNSKNTLYLQMNSLRAEDTAVYYCAKHGNFGNSYVSWFAYWGQGTLVTVSS

SEQ ID NO: 44 - protein, 125 aa, synthetic construct
  REGION 1..125: note = VH3231|VH|Chothia
EVQLLESGGGLVQPGGSLRLSCAASGFTFNTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSS

SEQ ID NO: 45 - protein, 125 aa, synthetic construct
  REGION 1..125: note = VH3232|VH|Chothia
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSS

SEQ ID NO: 46 - protein, 125 aa, synthetic construct
  REGION 1..125: note = VH3233|VH|Chothia
EVQLLESGGGLVQPGGSLRLSCAASGFTFSTYAMNWVRQAPGKGLEWVSRIRSKYNNYATYYADSVKDRFTISRDNSKNTLYLQMNSLRAEDTAVYYCAKHGNFGNSYVSWFAYWGQGTLVTVSS

SEQ ID NO: 47 - protein, 125 aa, synthetic construct
  REGION 1..125: note = VH3234|VH|Chothia
EVQLLESGGGLVQPGGSLRLSCAASGFTFSTYAMNWVRQAPGKGLEWVSRIRSKYNNYATYYADSVKDRFTISRDNSKNTLYLQMNSLRAEDTAVYYCVRHGNFGNSYVSWFAYWGQGTLVTVSS

SEQ ID NO: 48 - protein, 125 aa, synthetic construct
  REGION 1..125: note = VH3235|VH|Chothia
EVQLLESGGGLVQPGGSLRLSCAASGFTFSTYAMNWVRQAPGKGLEWVSRIRSKYNNYATYYADSVKDRFTISRDDSKSTLYLQMNSLRAEDTAVYYCAKHGNFGNSYVSWFAYWGQGTLVTVSS

SEQ ID NO: 49 - protein, 125 aa, synthetic construct
  REGION 1..125: note = VH3236|VH|Chothia
EVQLLESGGGLVQPGGSLRLSCAASGFTFSTYAMNWVRQAPGKGLEWVSRIRSKYNNYATYYADSVKDRFTISRDNSKSTLYLQMNSLRAEDTAVYYCARHGNFGNSYVSWFAYWGQGTLVTVSS

SEQ ID NO: 50 - protein, 125 aa, synthetic construct
  REGION 1..125: note = VH3730|VH|Chothia
EVQLVESGGGLVQPGGSLKLSCAASGFTFNTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 51 - protein, 125 aa, synthetic construct
  REGION 1..125: note = VH3731|VH|Chothia
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSS

SEQ ID NO: 52 - protein, 125 aa, synthetic construct
  REGION 1..125: note = VH3732|VH|Chothia
EVQLVESGGGLVQPGGSLKLSCAASGFTFSTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSS

SEQ ID NO: 53 - protein, 125 aa, synthetic construct
  REGION 1..125: note = VH3733|VH|Chothia
EVQLVESGGGLVQPGGSLKLSCAASGFTFSTYAMNWVRQASGKGLEWVGRIRSKYNNYATYYADSVKDRFTISRDDSKSTLYLQMNSLKTEDTAVYYCTRHGNFGNSYVSWFAYWGQGTLVTVSS

SEQ ID NO: 54 - protein, 125 aa, synthetic construct
  REGION 1..125: note = VH3734|VH|Chothia
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCVRHGNFGNSYVSWFAYWGQGTLVTVSS

SEQ ID NO: 55 - protein, 125 aa, synthetic construct
  REGION 1..125: note = VH3735|VH|Chothia
EVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 56 - protein, 109 aa, synthetic construct
  REGION 1..109: note = SP34_VL|VL|Chothia
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 57 - protein, 109 aa, synthetic construct
  REGION 1..109: note = VL7460|VL|Chothia
QAVVTQEPSLTVSPGGTVTLTCRSSTGAVTTSNYANWFQQKPGQAPRTLIYGTNKRAPWTPARFSGSLLGGKAALTLLGAQPEDEAEYYCALWYSNLWVFGGGTKLTVL

SEQ ID NO: 58 - protein, 109 aa, synthetic construct
  REGION 1..109: note = VL7461|VL|Chothia
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVL

SEQ ID NO: 59 - protein, 110 aa, synthetic construct
  REGION 1..110: note = VK1392|VL|Chothia
DIQMTQSPSSLSASVGDRVTITCRSSTGAVTTSNYANWYQQKPGKAPKLLIYGTNKRAPGVPSRFSGSGSGTDFTLTISSLQPEDFATYYCALWYSNLWVFGGGTKVEIK

SEQ ID NO: 60 - protein, 110 aa, synthetic construct
  REGION 1..110: note = VK1393|VL|Chothia
DAQMTQSPSSLSASVGDRVTITCRSSTGAVTTSNYANWVQQKPGKAPKGLIGGTNKRAPGVPSRFSGSLSGDDATLTISSLQPEDFATYFCALWYSNLWVFGGGTKVEIK

SEQ ID NO: 61 - protein, 107 aa, synthetic construct [sequence omitted]

SEQ ID NO: 62 - protein, 106 aa, synthetic construct
  REGION 1..106: note = human C_lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 63 - protein, 330 aa, synthetic construct
  REGION 1..330: note = human IgG1 heavy chain constant regions(with L234A, L235A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 330 aa, synthetic construct
  REGION 1..330: note = human IgG1 heavy chain constant regions(with L234A, L235A, P329G)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 15 aa, synthetic construct
  REGION 1..15: note = (G4S)3 linker
GGGGSGGGGSGGGGS

SEQ ID NO: 66 - protein, 20 aa, synthetic construct
  REGION 1..20: note = (G4S)4 linker
GGGGSGGGGSGGGGSGGGGS

SEQ ID NO: 67 - protein, 15 aa, synthetic construct
  REGION 1..15: note = (G4S)3 linker variant
GGGQSGQGGSGGGGS

SEQ ID NO: 68 - protein, 258 aa, synthetic construct
  REGION 1..258: note = synthetic antibody PR000275 anti-CD3e SP34_scFv_L3H-6His
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGSHHHHHH

SEQ ID NO: 69 - protein, 258 aa, synthetic construct
  REGION 1..258: note = synthetic antibody PR000276 anti-CD3e SP34_scFv_H3L-6His
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGSHHHHHH

SEQ ID NO: 70 - protein, 262 aa, synthetic construct
  REGION 1..262: note = synthetic antibody PR000307 anti-CD3e SP34_scFv_L4H-6His
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASHHHHHH

SEQ ID NO: 71 - protein, 262 aa, synthetic construct
  REGION 1..262: note = synthetic antibody PR000308 anti-CD3e SP34_scFv_H4L-6His
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLASHHHHHH

SEQ ID NO: 72 - protein, 483 aa, synthetic construct
  REGION 1..483: note = synthetic antibody PR000509 anti-CD3 SP34 hum-scFv(VH3731-GS-VL7461)-hFc(LALA)
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGQSGQGGSGGGGSQAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLASEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 483 aa, synthetic construct
  REGION 1..483: note = synthetic antibody PR000510 anti-CD3 SP34 hum-scFv(VL7461-GS-VH3731)-hFc(LALA)
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGGGQSGQGGSGGGGSEVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 484 aa, synthetic construct
  REGION 1..484: note = synthetic antibody PR000623 anti-CD3E SP34 hum-scFv(VH3231_GS_VK1393)-hFc(C220S,LALA)
EVQLLESGGGLVQPGGSLRLSCAASGFTFNTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSGGGQSGQGGSGGGGSDAQMTQSPSSLSASVGDRVTITCRSSTGAVTTSNYANWVQQKPGKAPKGLIGGTNKRAPGVPSRFSGSLSGDDATLTISSLQPEDFATYFCALWYSNLWVFGGGTKVEIKASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 484 aa, synthetic construct
  REGION 1..484: note = synthetic antibody PR000624 anti-CD3E SP34 hum-scFv(VH3731_GS_VK1393)-hFc(C220S,LALA)
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGQSGQGGSGGGGSDAQMTQSPSSLSASVGDRVTITCRSSTGAVTTSNYANWVQQKPGKAPKGLIGGTNKRAPGVPSRFSGSLSGDDATLTISSLQPEDFATYFCALWYSNLWVFGGGTKVEIKASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 484 aa, synthetic construct
  REGION 1..484: note = synthetic antibody PR000625 anti-CD3E SP34 hum-scFv(VK1393_GS_VH3231)-hFc(C220S,LALA)
DAQMTQSPSSLSASVGDRVTITCRSSTGAVTTSNYANWVQQKPGKAPKGLIGGTNKRAPGVPSRFSGSLSGDDATLTISSLQPEDFATYFCALWYSNLWVFGGGTKVEIKGGGQSGQGGSGGGGSEVQLLESGGGLVQPGGSLRLSCAASGFTFNTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 484 aa, synthetic construct
  REGION 1..484: note = synthetic antibody PR000626 anti-CD3E SP34 hum-scFv(VK1393_GS_VH3731)-hFc(C220S,LALA)
DAQMTQSPSSLSASVGDRVTITCRSSTGAVTTSNYANWVQQKPGKAPKGLIGGTNKRAPGVPSRFSGSLSGDDATLTISSLQPEDFATYFCALWYSNLWVFGGGTKVEIKGGGQSGQGGSGGGGSEVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 483 aa, synthetic construct
  REGION 1..483: note = synthetic antibody PR000627 anti-CD3E SP34 hum-scFv(VL7461_GS_VH3731)-hFc(C220S,LALA)
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGGGQSGQGGSGGGGSEVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 483 aa, synthetic construct
  REGION 1..483: note = synthetic antibody PR000914 anti-CD3E SP34 hum-scFv(VH3231_GS_VL7461)-hFc(C220S,LALA)
EVQLLESGGGLVQPGGSLRLSCAASGFTFNTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSGGGQSGQGGSGGGGSQAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 483 aa, synthetic construct
  REGION 1..483: note = synthetic antibody PR000915 anti-CD3E SP34 hum-scFv(VL7461_GS_VH3231)-hFc(C220S,LALA)
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGGGQSGQGGSGGGGSEVQLLESGGGLVQPGGSLRLSCAASGFTFNTYAMNWVRQAPGKGLEWVSRIRSKYNNYATYYADSVKDRFTISRDDSKSTLYLQMNSLRAEDTAVYYCVRHGNFGNSYVSWFAYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 483 aa, synthetic construct
  REGION 1..483: note = synthetic antibody PR001850 anti-CD3 SP34 hum-scFv(VL7461-GS-VH3732)-hFc(AAG)
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGGGQSGQGGSGGGGSEVQLVESGGGLVQPGGSLKLSCAASGFTFSTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 451 aa, synthetic construct
  REGION 1..451: note = synthetic antibody PR000014 B7H4 heavychain
EVQLVQSGAEVKKPGASVKVSCKASGYTFTSYWIGWVRQAPGQGLEWIGDIYPGGGYTNYNEKFKGRVTITRDTSTSTAYLELSSLRSEDTAVYYCARLAGSSYRGAMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 214 aa, synthetic construct
  REGION 1..214: note = synthetic antibody PR000014 B7H4 lightchain; PR002883 B7H4 lightchain
DIQMTQSPSSLSASVGDRVTITCKASQGFNKYVAWYQQKPGKAPKLLIYYTSTLQPGVPSRFSGSGSGRDYTLTISSLQPEDFATYYCLQYGDLLYAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 449 aa, synthetic construct
  REGION 1..449: note = synthetic antibody PR000374 ROR1 heavychain
EVQLVESGGGLVQPGRSLRLSCTASGSDINDYPITWVRQAPGQGLEWIGFINSGGSTWYASWVKGRFTISRDDSKSIAYLQMNSLKTEDTAVYYCARGYSTYYRDFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 217 aa, synthetic construct
  REGION 1..217: note = synthetic antibody PR000374 ROR1 lightchain; PR002885 ROR1 lightchain
DIQMTQSPSSLSASVGDRVTINCQASQSIDSNLAWFQQKPGQPPKLLIYRASNLASGVPDRFSGSGSGTDFTLTISSLEAEDVATYYCLGGVGAVSYRTSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 451 aa, synthetic construct
  REGION 1..451: note = synthetic antibody PR002883 B7H4 Hole-heavychain
EVQLVQSGAEVKKPGASVKVSCKASGYTFTSYWIGWVRQAPGQGLEWIGDIYPGGGYTNYNEKFKGRVTITRDTSTSTAYLELSSLRSEDTAVYYCARLAGSSYRGAMDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 87 - protein, 449 aa, synthetic construct
  REGION 1..449: note = synthetic antibody PR002885 ROR1 Hole-heavychain
EVQLVESGGGLVQPGRSLRLSCTASGSDINDYPITWVRQAPGQGLEWIGFINSGGSTWYASWVKGRFTISRDDSKSIAYLQMNSLKTEDTAVYYCARGYSTYYRDFNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 88 - protein, 483 aa, synthetic construct
  REGION 1..483: note = synthetic antibody PR002883 chain-2;synthetic antibody PR002885 chain-2; (synthetic antibody PR000627 in knob-Fc)
QAVVTQEPSLTVSPGGTVTLTCRSSTGAVTTSNYANWVQQKPGQAPRGLIGGTNKRAPWTPARFSGSLLGDKAALTLLGAQPEDEAEYFCALWYSNLWVFGGGTKLTVLGGGQSGQGGSGGGGSEVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 117 aa, synthetic construct
  REGION 1..117: note = IGHV3-73*01|GHJ1*01
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTRAEYFQHWGQGTLVTVSS

SEQ ID NO: 90 - protein, 115 aa, synthetic construct
  REGION 1..115: note = IGHV3-23*01|IGHJ1*01
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAEYFQHWGQGTLVTVSS

SEQ ID NO: 91 - protein, 110 aa, synthetic construct
  REGION 1..110: note = IGLV7-46*02|IGLJ2*01
QAVVTQEPSLTVSPGGTVTLTCGSSTGAVTSGHYPYWFQQKPGQAPRTLIYDTSNKHSWTPARFSGSLLGGKAALTLLGAQPEDEAEYYCLLSYSGARVVFGGGTKLTVL

SEQ ID NO: 92 - protein, 107 aa, synthetic construct [sequence omitted]